(19) United States
(12) Patent Application Publication
(10) Pub. No.: US 2017/0218324 A1
(43) Pub. Date: Aug. 3, 2017

(54) CELL CULTURE DEVICE, METHOD OF FORMING THE SAME, AND METHOD OF PARTIALLY DETACHING STEM CELLS FROM THE SAME

(71) Applicants: NATIONAL CENTRAL UNIVERSITY, TAOYUAN CITY (TW); CHUNG YUAN CHRISTIAN UNIVERSITY, TAOYUAN CITY (TW)

(72) Inventors: Akon HIGUCHI, TAOYUAN CITY (TW); Yung CHANG, Taoyuan City (TW); Hsing-Fen LI, TAOYUAN CITY (TW)

(21) Appl. No.: 15/009,845

(22) Filed: Jan. 29, 2016

(51) Int. Cl. C12M 1/00; C12M 1/26

(52) U.S. Cl. CPC C12M 47/04; C12M 33/08; C12M 47/20

(57) ABSTRACT

A cell culture device includes a container having a surface and a plurality of nano-bristles immobilized to the surface. The nano-bristles are made of styrene copolymer or oligopeptide. A method of partially detaching stem cells from a cell culture device includes: seeding stem cells onto the plurality of nano-bristles, culturing the stem cells, detaching a portion of the stem cells from the nano-bristles by a change in temperature or a shear force, and harvesting the portion of the stem cells.

### BACKGROUND

Field of Disclosure

The present disclosure relates to a cell culture device.

Description of Related Art

Current stem cell culture in two dimensional (e.g. flask and dish culture) and three dimensional culture (e.g. microcarrier culture) is the batch type process. In these culture systems, stem cell culture processes have three steps: cell seeding and attachment to the surface, cell expansion, and cell detachment. To obtain large numbers of stem cells for cell therapy, these steps must be performed repeatedly. In most cases, enzymes, such as trypsin for human adult stem cells (hASCs), and dispase or accutase for human embryonic stem cells (hESCs) and human induced pluripotent stem cells (hiPSCs), are used to detach cells from cell culture dishes or microcarriers. Nonetheless, the enzymatic treatment has been found to ruin cell membrane by hydrolyzing some membrane-associated proteins and disrupting both the cell junction and extracellular matrix (ECM), which impairs cell function and makes cell culture dishes and microcarriers disposable.

Furthermore, while some dishes and microcarriers are used in conventional stem cell culture to detach stem cells without enzyme treatments, total detachment occurs to the whole sheet of cells, rendering no stem cells to remain and expand on the surface. To harvest large numbers of stem cells, the cells must be seeded repeatedly. Therefore, large numbers of dishes and microcarriers have been required for stem cells expansion in conventional stem cell culture methods due to inability of the partial detachment of stem cells.

To solve bottlenecks of the conventional stem cell culture system, a new stem cell culture system facilitating the partial detachment and thus the continuous harvesting of stem cells is of great needs.

### SUMMARY

The present disclosure provides a cell culture device including a container having a surface and a plurality of nano-bristles immobilized to the surface. The nano-bristles are made of styrene copolymer or oligopeptide.

According to some embodiments of the present disclosure, the surface contains polystyrene, and the nano-bristles are made of the styrene copolymer, and the styrene copolymer includes poly[styrene-co-N-isopropylacrylamide](P[St-NIPAAm]) and poly[styrene-co-acrylic acid] (P[St-AA]).

According to some embodiments of the present disclosure, the cell culture device further includes an extracellular matrix (ECM) segment grafted to the P[St-AA], and the P[St-AA] is between the ECM segment and the surface containing polystyrene.

According to some embodiments of the present disclosure, the ECM segment is vitronectin (VN) oligopeptide.

According to some embodiments of the present disclosure, the styrene copolymer further includes poly[styrene-co-polyethylene glycol methacrylate](P[St-PEGMA]).

According to some embodiments of the present disclosure, a weight ratio of the P[St-PEGMA] to the P[St-NIPAAm] is 0:1 to 3:7.

According to some embodiments of the present disclosure, the surface contains polyvinylalcohol-co-itaconic acid (PVA-IA) hydrogel, and the nano-bristles are made of the oligopeptide, and the oligopeptide includes vitronectin (VN) oligopeptide, bone sialprotein (BSP) oiligopeptide, heparin binding peptide (HBP1), vitronectin-2C (VN2C) oligopeptide, vitronectin-2G (VN2G) oligopeptide, or a combination thereof.

According to some embodiments of the present disclosure, the PVA-IA hydrogel has a storage modulus of 10-35 kPa.

According to some embodiments of the present disclosure, the container includes a dish, a flask, or a microcarrier.

The present disclosure provides a method of forming a cell culture device. The method includes: providing a container having a surface which contains polystyrene, coating poly[styrene-co-acrylic acid] (P[St-AA]) onto the surface, activating the surface coated with the P[St-AA] by N-(3-dimethylaminopropyl)-N′-ethylcarbodiimide hydrochloride (EDC) and N-hydroxysuccinimide (NHS) to form an activated surface, grafting an extracellular matrix (ECM) segment to the P[St-AA] after activating the surface coated with the P[St-AA], and coating poly[styrene-co-N-isopropylacrylamide](P[St-NIPAAm]) onto the activated surface after grafting the ECM segment.

According to some embodiments of the present disclosure, a method of forming a cell culture device further includes coating poly[styrene-co-polyethylene glycol methacrylate] (P[St-PEGMA]) onto the activated surface after grafting the ECM segment.

According to some embodiments of the present disclosure, coating the P[St-AA] onto the surface includes adding a first solution containing the P[St-AA]onto the surface, and the P[St-AA] has a concentration of 0.5-3.5 mg/ml in the first solution.

According to some embodiments of the present disclosure, coating the P[St-NIPAAm] onto the activated surface includes adding a second solution containing the P[St-NIPAAm] onto the activated surface, and the P[St-NIPAAm] has a concentration of 2-5 mg/ml in the second solution.

According to some embodiments of the present disclosure, coating the P[St-PEGMA] onto the activated surface includes adding a third solution containing the P[St-PEGMA] onto the activated surface, and the P[St-PEGMA] has a concentration of 2-5 mg/ml in the third solution.

The present disclosure also provides a method of forming a cell culture device. The method includes: providing a container having a surface which contains polyvinylalcohol-co-itaconic acid (PVA-IA) hydrogel, activating the PVA-IA hydrogel with N-(3-dimethylaminopropyl)-N′-ethylcarbodiimide hydrochloride (EDC) and N-hydroxysuccinimide (NHS) to form activated PVA-IA hydrogel, and grafting oligopeptide to the activated PVA-IA hydrogel.

According to some embodiments of the present disclosure, grafting the oligopeptide to the activated PVA-IA hydrogel includes adding a fourth solution containing the oligopeptide onto the activated PVA-IA hydrogel, and the oligopeptide has a concentration of 40-1600 μg/ml in the fourth solution.

The present disclosure also provides a method of partially detaching stem cells from a cell culture device. The method includes: seeding stem cells onto the plurality of nano-bristles, culturing the stem cells, detaching a portion of the stem cells from the nano-bristles by a change in temperature or a shear force, and harvesting the portion of the stem cells.

According to some embodiments of the present disclosure, the nano-bristles are made of the styrene copolymer; and detaching the portion of the stem cells from the nano-bristles by the change in the temperature includes lowering a temperature of the nano-bristles to 0-20° C.

According to some embodiments of the present disclosure, the nano-bristles are made of the oligopeptide, and detaching the portion of the stem cells from the nano-bristles by the shear force includes shaking the nano-bristles.

According to some embodiments of the present disclosure, the stem cells include human adipose-derived stem cells (hADSCs), human embryonic stem cells (hESCs), human induced pluripotent stem cells (hiPSCs), or a combination thereof.

It is to be understood that both the foregoing general description and the following detailed description are by examples, and are intended to provide further explanation of the present disclosure as claimed.

### BRIEF DESCRIPTION OF THE DRAWINGS

The present disclosure can be more fully understood by reading the following detailed description of the embodiment, with reference made to the accompanying drawings as follows:

FIG. 1A illustrates a cross section view of a cell culture device in accordance with some embodiments of the present disclosure.

FIG. 1B illustrates a cross section view of a cell culture device in accordance with some embodiments of the present disclosure.

FIG. 2 illustrates a flow chart of forming a cell culture device in accordance with some embodiments of the present disclosure.

FIG. 3A illustrates a cross section view of a cell culture device in accordance with some embodiments of the present disclosure.

FIG. 3B illustrates a cross section view of a cell culture device in accordance with some other embodiments of the present disclosure.

FIG. 4 illustrates a flow chart of forming a cell culture device in accordance with some embodiments of the present disclosure.

FIG. 5 illustrates a flow chart of partially detaching stem cells from a cell culture device in accordance with some embodiments of the present disclosure.

FIG. 6 illustrates morphologies of hADSCs cultured on thermoresponsive surfaces in accordance with Experimental examples 1-3 and Comparative examples 1-2 of the present disclosure.

FIG. 7 illustrates morphologies of hADSCs throughout cycles of partial detachment in accordance with Experimental example 2 of the present disclosure.

FIG. 8 illustrates detachment ratios of hADSCs throughout cycles of partial detachment in accordance with Experimental examples 1-3 of the present disclosure.

FIG. 9 illustrates five/dead staining of hADSCs after cycles of partial detachment in accordance with Experimental example 2 of the present disclosure.

FIG. 10A-10D illustrates flow cytometry scattergrams of stem cell marker expressions in hADSCs after cycles of partial detachment in accordance with Experimental example 2 of the present disclosure.

FIG. 11 illustrates morphologies of hESCs cultured on thermoresponsive surfaces in accordance with Experimental examples 1-2 and Comparative examples 3-4 of the present disclosure.

FIG. 12A illustrates attachment ratios of hESCs cultured on thermoresponsive surfaces in accordance with Experimental examples 1-2, 4-13 and Comparative examples 1, 4-7 of the present disclosure.

FIG. 12B illustrates detachment ratios of hESCs cultured on thermoresponsive surfaces in accordance with Experimental examples 1-2, 4-13 and Comparative examples 1, 4-7 of the present disclosure.

FIG. 12C illustrates differentiation ratios of hESCs cultured on thermoresponsive surfaces in accordance with Experimental examples 1-2, 4-13 and Comparative examples 1, 4-7 of the present disclosure.

FIG. 13A illustrates morphologies of hESCs throughout cycles of partial detachment in accordance with Experimental example 11 of the present disclosure.

FIG. 13B illustrates detachment ratios of hESCs throughout cycles of partial detachment in accordance with Experimental example 11 and Comparative example 4 of the present disclosure.

FIG. 14A illustrates immunostaining images of pluripotency marker expressions in hESCs after cycles of partial detachment in accordance with Experimental example 11 of the present disclosure.

FIG. 14B illustrates embryoid bodies formed by hESCs after cycles of partial detachment in accordance with Experimental example 11 of the present disclosure.

FIG. 14C illustrates immunostaining images of differentiation marker expressions in hESCs after cycles of partial detachment in accordance with Experimental example 11 of the present disclosure.

FIG. 15A-15D illustrates high-resolution XPS spectra of C1s and N1s peaks in accordance with Experimental example 14 and Comparative example 8 of the present disclosure.

FIG. 16A illustrates atomic ratios of nitrogen to carbon (N/C) of PVA-IA-oligoVN hydrogels under different oligoVN concentrations in accordance with Experimental examples 14-19 and Comparative examples 2, 9 of the present disclosure.

FIG. 16B illustrates atomic ratios of nitrogen to carbon (N/C) of PVA-IA-oligoVN hydrogels under different lengths of crosslinking time in accordance with Experimental examples 18, 20-23 of the present disclosure.

FIG. 17A illustrates morphologies of hESCs cultured on PVA-IA-oligoVN hydrogels with different elasticities in accordance with Experimental examples 18, 20-23 and Comparative examples 4, 10 of the present disclosure.

FIG. 17B illustrates attachment ratios of hESCs and hiPSCs cultured on PVA-IA-oligoVN hydrogels with different elasticities in accordance with Experimental examples 18, 21-23 and Comparative examples 4, 10 of the present disclosure.

FIG. 17C illustrates differentiation ratios of hESCs and hiPSCs cultured on PVA-IA-oligoVN hydrogels with different elasticities in accordance with Experimental examples 18, 21-23 and Comparative examples 4, 10 of the present disclosure.

FIG. 18A illustrates morphologies of hESCs cultured on PVA-IA-oligoVN hydrogels with different surface densities of oligoVN in accordance with Experimental examples 14-19 and Comparative examples 4, 10 of the present disclosure.

FIG. 18B illustrates attachment ratios of hESCs and hiPSCs cultured on PVA-IA-oligoVN hydrogels with different surface densities of oligoVN in accordance with Experimental examples 14, 16-19 and Comparative examples 4, 10 of the present disclosure.

FIG. 18C illustrates differentiation ratios of hESCs and hiPSCs cultured on PVA-IA-oligoVN hydrogels with different surface densities of oligoVN in accordance with Experimental examples 14, 16-19 and Comparative examples 4, 10 of the present disclosure.

FIG. 19A-19B illustrates expansion rates of hESCs and hiPSCs cultured on PVA-IA-oligoVN hydrogels with an optimal elasticity throughout passages in accordance with Experimental example 14 and Comparative examples 4, 10 of the present disclosure.

FIG. 20A-20B illustrates attachment ratios of hESCs and hiPSCs cultured on PVA-IA-oligoVN hydrogels with an optimal elasticity throughout passages in accordance with Experimental example 14 and Comparative examples 4, 10 of the present disclosure.

FIG. 21A-21B illustrates differentiation ratios of hESCs and hiPSCs cultured on PVA-IA-oligoVN hydrogels with an optimal elasticity throughout passages in accordance with Experimental example 14 and Comparative examples 4, 10 of the present disclosure.

FIG. 22 illustrates colony morphologies of hESCs cultured on PVA-IA-oligoVN hydrogels with an optimal elasticity in accordance with Experimental example 14 and Comparative example 10 of the present disclosure.

FIG. 23A illustrates immunostaining images of pluripotency marker expressions of hESCs cultured on PVA-IA-oligoVN hydrogels in accordance with Experimental example 14 of the present disclosure.

FIG. 23B illustrates immunostaining images of pluripotency marker expressions of hiPSCs cultured on PVA-IA-oligoVN hydrogels in accordance with Experimental example 14 of the present disclosure.

FIG. 24A illustrates embryoid bodies formed by hESCs and hiPSCs cultured on PVA-IA-oligoVN hydrogels in accordance with Experimental example 14 of the present disclosure.

FIG. 24B illustrates immunostaining images of differentiation marker expressions of hESCs cultured on PVA-IA-oligoVN hydrogels in accordance with Experimental example 14 of the present disclosure.

FIG. 24C illustrates immunostaining images of differentiation marker expressions of hiPSCs cultured on PVA-IA-oligoVN hydrogels in accordance with Experimental example 14 of the present disclosure.

FIG. 25A-25D illustrates in vivo differentiation of hESCs cultured on PVA-IA-oligoVN hydrogels in accordance with Experimental example 14 of the present disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

As aforementioned, the enzymatic treatment to detach stem cells has been found to cause damage to the cell membrane. As for some surfaces from which stem cells can detach without enzyme treatments, total detachment usually occurs to the complete cell sheet, which involves water penetration from the periphery and/or the bottom of the surface, rendering no stem cells attached to the surface. Hence, both ways mentioned above cost not only great amount of disposable dishes, flasks or microcarriers, but also great effort and time for repeated seeding of stem cells in order to harvest a large amount for clinical use due to the inability of partial detachment of stem cells.

Therefore, the present disclosure provides a cell culture device to facilitate the partial detachment and continuous harvesting of stem cells by utilizing the nano-bristles on a surface of the cell culture device. With merely changing the temperature or exerting a shear force to the nano-bristles, stem cells can partially detach from the nano-bristles, enabling the elimination of the enzyme treatment and further expansion of the attached cells in the same cell culture device, which facilitates continuous harvesting of the stem cells.

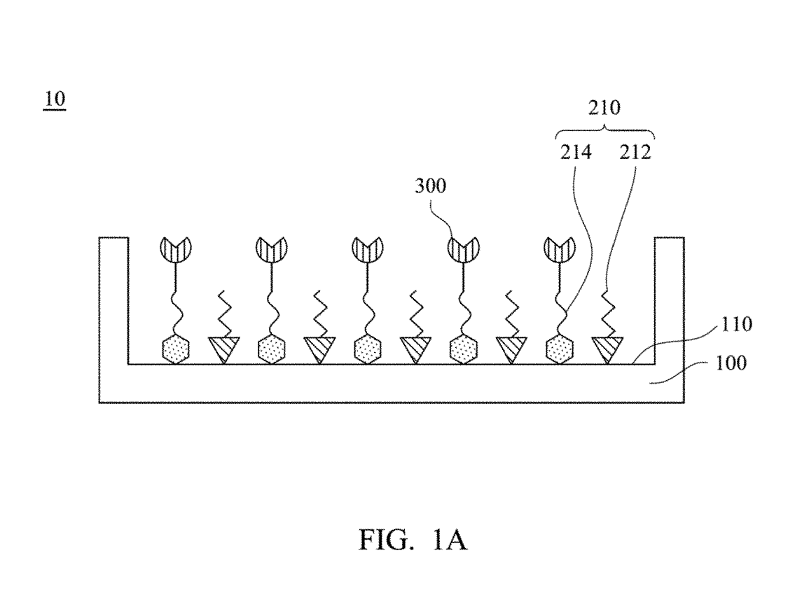

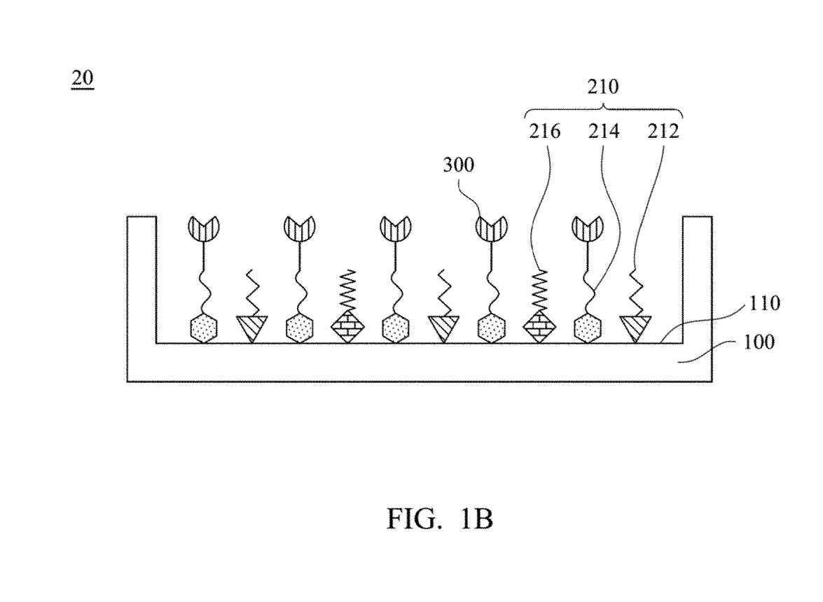

Referring to FIG. 1A, which illustrates a cross section view of a cell culture device in accordance with some embodiments of the present disclosure. The cell culture device 10 includes a container 100 having a surface 110, and a plurality of nano-bristles 200 immobilized to the surface 110. The nano-bristles 200 are made of styrene copolymer 210 or oligopeptide 220.

In some embodiments, the surface 110 contains polystyrene, the nano-bristles 200 are made of styrene copolymer 210, and the styrene copolymer 210 includes poly[styrene-co-N-isopropylacrylamide] (P[St-NIPAAm]) 212 and poly[styrene-co-acrylic acid] (P[St-AA]) 214. In those embodiments, both the surface 110 and the styrene copolymer 210 contain polystyrene, which facilitates the binding of the styrene copolymer 210 to the surface 110. In other words, the hydrophobic polystyrene (PSt) serves as an anchor for the styrene copolymer 210 to be coated onto the surface 110.

The P[St-NIPAAm] 212 contains N-isopropylacrylamide] (PNIPAAm), which is characterized for the thermoresponsiveness. The PNIPAAm includes repeating units of hydrophilic amide groups and hydrophobic isopropyl groups. When the temperature rises, especially above the lower critical solution temperature (LCSC), the PNIPAAm tends to transform from a hydrophilic, expanded coil to a hydrophobic, compact globule. Hence, the P[St-NIPAAm] 212 exhibits hydrophobicity at a higher temperature, such as at 35-40° C., which enhances cell binding due to the hydrophobicity of the cell membrane. However, with the decrease of temperature, the hydrophobicity of the P[St-NIPAAm] 212 also decreases. The lower the hydrophobicity, the higher the hydrophilicity. The increased hydrophilicity of the P[St-NIPAAm] 212 at lower temperature, such as at 0-20° C., is unfavorable for cell adhesion, and contributes to cell detachment.

The P[St-AA] 214 contains polyacrylic acid (PAA), which provides binding sites for an extracellular matrix (ECM) segment 300 to facilitate stem cell binding. In some embodiments, the ECM segment 300 is grafted to the P[St-AA] 214, and the P[St-AA] 214 is between the ECM segment 300 and the surface 110 containing polystyrene. To put it differently, the ECM segment 300 might not be in direct contact with the surface 110, and thus the P[St-AA] 214 serves as a bridge between the ECM segment 300 and the surface 110. In some embodiments, the extracellular matrix (ECM) segment 300 is vitronectin (VN) oligopeptide (herein abbreviated as oligoVN) with the amino acid sequence of KGGPQVTRGDVFTMP). Stem cells cultured on surfaces with grafted oligoVN are reported to well maintain pluripotency, especially for human embryonic stem cells (hESCs) and human induced pluripotent stem cells (hiPSCs).

Referring to FIG. 1, which illustrates a cross section view of a cell culture device in accordance with some embodiments of the present disclosure. As shown in FIG. 1B, the styrene copolymer 210 further includes poly[styrene-co-polyethylene glycol methacrylate] (P[St-PEGMA]) 216. The P[St-PEGMA] 216 contains polyethylene glycol methacrylate] (PEGMA), which is highly hydrophilic. In some embodiments the coating of the hydrophilic P[St-PEGMA] 216 to the surface 110 accelerates the cell detachment at low temperatures. In some embodiments, a weight ratio of the P[St-PEGMA] 216 to the P[St-NIPAAm] 212 is 0:1 to 3:7.

In some embodiments, due to the assistance in providing more hydrophilicity, merely lowering the temperature is enough for cells to detach from the nano-bristles 200. Since there is no need for water penetration from the periphery and/or the bottom of the surface, the complete detachment of the whole cell sheet can be avoided, and partial detachment of stem cells from the cell culture device can be achieved.

Coating the styrene copolymer 210 with distinct concentration or weight ratios of the P[St-NIPAAm] 212, P[St-AA] 214, and P[St-PEGMA] 216 onto the surface 110 containing polystyrene gives rise to nano-bristles with varying compositions and affinities for hASCs, hESCs, and hiPSCs. In some embodiments, when the nano-bristles 200 are made of the styrene copolymer 210, the way of partially detaching stem cells from the surface is mainly attributed to the thermoresponsiveness of the P[St-NIPAAm]. Hence, in those embodiments, the surface 110 along with the coated styrene copolymer 210 can be collectively referred to as a thermoresponsive surface for simplicity.

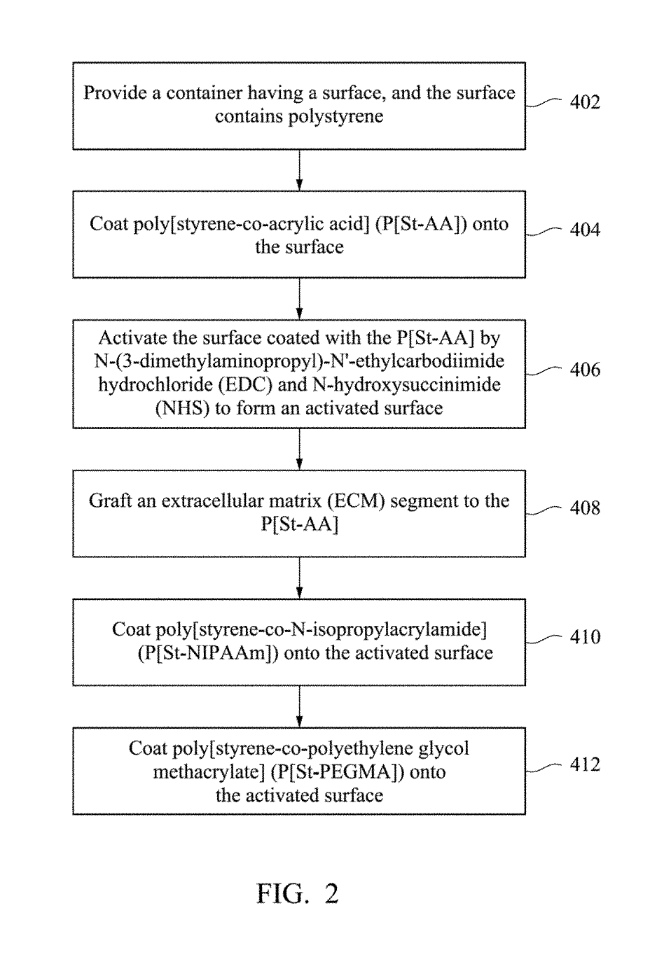

Referring to FIG. 2, which illustrates a flow chart of forming a cell culture device in accordance with some embodiments of the present disclosure. The method includes: providing a container 100 having a surface 110, and the surface 110 contains polystyrene (step 402), coating poly[styrene-co-acrylic acid] (P[St-AA]) 214 onto the surface 110 (step 404), activating the surface 110 coated with the P[St-AA] 214 by N-(3-dimethylaminopropyl)-N′-ethylcarbodiimide hydrochloride (EDC) and N-hydroxysuccinimide (NHS) to form an activated surface (step 406), grafting an extracellular matrix (ECM) segment 300 to the P[St-AA] 214 after activating the surface coated with the P[St-AA] 214 (step 408), and coating poly[styrene-co-N-isopropylacrylamide] (P[St-NIPAAm]) 212 onto the activated surface after grafting the ECM segment 300 (step 410). The method further includes coating poly[styrene-co-polyethylene glycol methacrylate](P[St-PEGMA]) 416 onto the activated surface after grafting the ECM segment 300 (step 412), which is only performed in forming the cell culture device 20 but not the cell culture device 10. The details of each step are elaborated below.

In step 402, the container 100 having the surface 110 is provided, and the surface 110 contains polystyrene to facilitate coating of the styrene copolymer 210 onto the surface. In some embodiments, the container is a dish, a flask, or a microcarrier. In some embodiments, the container is a tissue culture polystyrene dish (TCPS).

Before coating the styrene copolymer 210 onto the surface 110, synthesis of the styrene copolymer 210 is performed. The P[St-AA] 214, P[St-NIPAAm]), 212 and P[St-PEGMA] 216 are synthesized by the reversible addition-fragmentation chain transfer (RAFT) polymerization. In detail, styrene monomers are radically polymerized with 5-cyano-5-[(phenyl-carbonothioyl)thio]hexanoic acid using 4,4′-azobis(4-cyanovaleric acid) in toluene to prepare a polystyrene-RAFT reagent at 70-90° C. for 14-18 hours. After recrystallization in hexane, the polystyrene-RAFT reagent is copolymerized with acrylic acid (AA) to form P[St-AA] 214, N-isopropylacrylamide (NIPAAm) to form P[St-NIPAAm] 212, or polyethylene glycol methacrylate (PEGMA) to form the P[St-PEGMA] 216. The copolyemerization is performed by 4,4′-azobis(4-cyanovaleric acid) in toluene at 70-90° C. for 14-18 hours. After recrystallization in hexane, the styrene copolymers 210 are freeze-dried and kept at 0-10° C. under dry conditions.

In some embodiments, for a single molecule of P[St-NIPAAm] 212, the number of repeating units of the PSt is 25-35, and the number of repeating units of the PAA units is 160-200. In some embodiments, for a single molecule of P[St-AA] 214, the number of repeating units of the PSt is 50-60, and the number of repeating units of the PAA units is 50-250. In some embodiments, for a single molecule of P[St-PEGMA] 216, the number of repeating units of the PSt is 55-65, and the number of repeating units of the PAA units is 100-250.

In step 404, coating the P[St-AA] 214 onto the surface includes adding a first solution containing the P[St-AA] 214 onto the surface, and the P[St-AA] 214 has a concentration of 0.5-3.5 mg/ml in the first solution. The P[St-AA] 214 (especially the P[St-AA] 214 with 170-180 repeating units of PAA) is dissolved in ethanol to form the first solution. The surface 110 is immersed in the first solution and incubated at 20-30° C. for 1-4 hours. After that, the first solution is subsequently removed, and the surface 110 coated with the P[St-AA] 214 is washed with phosphate-buffered saline (PBS, pH 7.2) for 2-5 times.

Next, in step 406, the-surface-coated with the P[St-AA] 214 is activated by immersion in an aqueous solution containing 5-20 mg/ml N-(3-dimethylaminopropyl)-N′-ethylcarbodiimide hydrochloride (EDC) and 5-20 mg/ml N-hydroxysuccinimide (NHS) for 0.5-2 hours at 30-40° C. In some embodiments, the EDC and the NHS are of the same concentration in the aqueous solution. EDC is a crosslinking agent utilized to couple carboxyl or phosphate groups to primary amines, which forms amide bonds. Nevertheless, the reactive intermediate formed in the coupling reaction can be rapidly hydrolyzed in aqueous solutions. Hence, to increase the stability of the intermediate, NHS is introduced to convert the amine-reactive intermediate into an NHS ester, which is more stable for subsequent coupling reactions. Subsequently, the activated surface containing the NHS ester is then washed with PBS for 2-5 times.

In step 408, the ECM segment 300 is grafted to the P[St-AA] 214. The grafting of the ECM segment 300 provides binding sites for stem cells, and thus enhances stem cell attachment. In some embodiments, the ECM segment 300 is the oligoVN. The ECM segment 300 is dissolved in the PBS solution and has a concentration of 40-1200 μg/ml in the PBS solution. For hADSC attachment and culture, the concentration of the ECM segment is 40-60 μg/ml. For hESC attachment and culture, the concentration of the ECM segment is 900-1200 μg/mi. The activated, P[St-AA]-coated surface is immersed in the PBS solution containing the ECM segment 300 for 20-28 hours at 0-10° C. to graft the ECM segment 300 to the P[St-AA] 214.

In step 410, coating the P[St-NIPAAm] 212 onto the activated surface includes adding a second solution containing the P[St-NIPAAm] 212 onto the activated surface, and the P[St-NIPAAm] 212 has a concentration of 2-5 mg/ml in the second solution. The P[St-NIPAAm]) 212 is dissolved in ethanol to form the second solution. The activated surface is immersed in the second solution and incubated for 1-4 hours at 20-30° C. After that, the second solution is subsequently removed, and the activated surface coated with the P[St-NIPAAm]212 is washed with PBS for 2-5 times. The coating of the P[St-NIPAAm] 212 provides thermoresponsiveness, which enables stem cells to detach at a lower temperature.

In step 412, coating the P[St-PEGMA] 216 onto the activated surface includes adding a third solution containing the P[St-PEGMA] 216 onto the activated surface, and the P[St-PEGMA] 216 has a concentration of 2-5 mg/ml in the third solution. The P[St-PEGMA]) 216 is dissolved in ethanol to form the third solution. The activated surface is immersed in the third solution and incubated for 1-4 hours at 20-30° C. After that, the third solution is subsequently removed, and the activated surface coated with the P[St-PEGMA]) 216 is washed with PBS for 2-5 times. The coating of the P[St-PEGMA]) 216 provides hydrophilicity, which enables the acceleration of stem cell detachment at a lower temperature.

In some embodiments, the second solution and the third solution are of the same concentration, and can be blended in varying volume ratios to form the activated surface coated with the varying weight ratios of the P[St-PEGMA]216 to the P[St-NIPAAm] 212, giving rise to varying partial detachment effects of stem cells. In some embodiments, the weight ratio of the P[St-PEGMA] 216 to the P[St-NIPAAm] 212 is 0:1 to 3:7, which includes 0:1, 1:9, 1:4, 3:7, and other appropriate ratios.

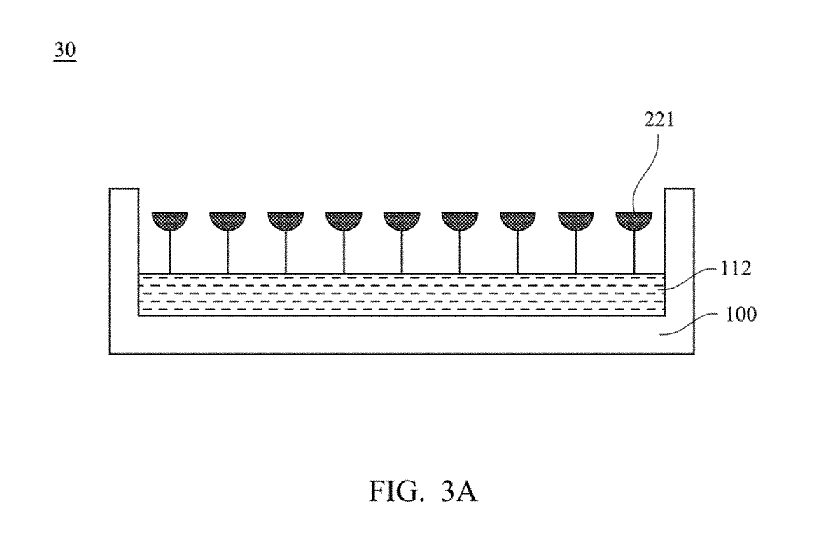

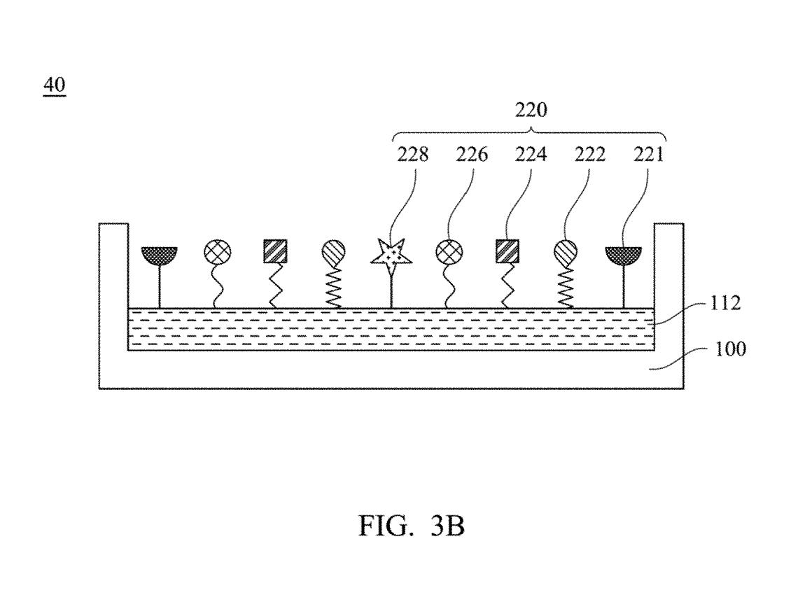

Referring next to FIG. 3A, which Illustrates a cross section view of a cell culture device in accordance with some embodiments of the present disclosure. The cell culture device 30 includes a container 100 having a surface 110. In some embodiments, the surface 110 contains polyvinylalcohol-co-itaconic acid (PVA-IA) hydrogel 112, and the nano-bristles 200 are made of oligopeptide 220, and the oligopeptide includes vitronectin (VN) oligopeptide 221 (also abbreviated as oligoVN), bone sialprotein (BSP) oligopeptide 222, heparin binding peptide (HBP1) 224, vitronectin-2C (VN2C) oligopeptide 226, vitronectin-2G (VN2G) oligopeptide 228, or a combination thereof.

The grafted oligopeptide provides stem cell binding sites and facilitates stem cell attachment, while the PVA-IA hydrogels 112 with different elasticity (i.e. storage modulus, E′) can facilitate partial detachment of stem cells upon the exertion of a shear force. In some embodiments, the PVA-IA hydrogel 112 has a storage modulus of 10-35 kPa.

The PVA-IA hydrogels 112 is characterized for varying elasticity according to varying crosslinking intensities. Crosslinking intensities rely mainly on the crosslinking time. Crosslinking of the PVA-IA hydrogels 112 can be carried out by agents such as glutaraidehyde, acetaldehyde, formaldehyde, and other monoaldehydes, which form acetal bridges between the pendant hydroxyl groups of the PVA chains. The crosslinked PVA-IA forms a dense structure with strong intra- and intermolecular hydrogen bonding and great amounts of carboxylate groups. The carboxylate group not only facilitates hydration of the PVA-IA hydrogels 112, not also provides some site for oligopeptides grafting.

Referring next to FIG. 3B, which illustrates a cross section view of a cell culture device in accordance with some embodiments of the present disclosure. The difference between the cell culture device 40 in FIG. 3B and the cell culture device 30 in FIG. 3A is the diversity of the grafted oligopeptides. In some embodiments, only one kind of oligopeptide is grafted to the PVA-IA hydrogel 112, like the cell culture device 30 shown in FIG. 3A. In some other embodiments, two or more kinds of oligopeptides are grafted to the PVA-IA hydrogel 112, like the cell culture device 40 shown in FIG. 3B. In some embodiments, the PVA-IA hydrogel 112 along with the grafted oligopeptide 220 can be collectively referred to as a PVA-IA-oligopeptide hydrogel for simplicity.

The oligo VN 221 has the amino acid sequence of KGGPQVTRGDVFTMP and serves as a crucial stem cell binding site to maintain the pluripotency of stem cells. The oligo VN 221 contains an RGD amino acid sequence, which is a binding site for the membrane-bound integrins, especially the integrins α _{V}β₃ and α_{V}β₅. Once the oligo VN 221 is grafted to the PVA-IA hydrogel 112, the binding of the oligo VN 221 to integrins anchors stem cells to the PVA-IA hydrogel 112.

The BSP oligopeptide 222 has the amino acid sequence of KGGNGEPRGDTYRAY and also contains the RGD sequence to bind to integrins. Once the BSP oligopeptide 222 is grafted to the PVA-IA hydrogel 112, the binding of the BSP oligopeptide 222 to integrins anchors cells to the PVA-IA hydrogel 112 and helps to maintains the pluripotency of stem cells.

The HBP1 224 has the amino acid sequence of GKKQRFRHRNRKG and contains a binding site recognized by heparan sulfate proteoglycans on the cell surface. The heparan sulfate proteoglycans are involved in focal adhesion of cells, which is a crucial feature for cell attachment. Thus, once the HBP1 224 is grafted to the PVA-IA hydrogel 112, the binding of the HBP1 224 to heparan sulfate proteoglycans anchors cells to the PVA-IA hydrogel 112 and helps to maintains the pluripotency of stem cells.

The VN2C oligopeptide 226 (with the amino acid sequence of GCGGKGGPQVTRGDVFTMP) is a dual chain molecule, with the dual chains connected by a disulfide bond. The sequence of the VN2C oligopeptide 226 differs from the oligoVN 221 by the addition of the GCGG sequence. Also containing the RGD sequence, VN2C oligopeptide 226 can thus bind to integrins. Once the B VN2C oligopeptide 226 is grafted to the PVA-IA hydrogel 112, the binding of the VN2C oligopeptide 226 to integrins anchors cells to the PVA-IA hydrogel 112 and helps to maintains the pluripotency of stem cells.

The VN2G oligopeptide 228 (with the amino acid sequence of GGGGKGGPQVTRGDVFTMP) is a dual chain molecule, with the dual chains connected by a disulfide bond. The sequence of the VN2G oligopeptide 228 differs from the oligoVN 221 by the addition of the GGGG sequence. Also containing the RGD sequence, VN2G oligopeptide 228 can thus bind to integrins. Once the VN2G oligopeptide 228 is grafted to the PVA-IA hydrogel 112, the binding of the VN2G oligopeptide 228 to integrins anchors cells to the PVA-IA hydrogel 112 and helps to maintains the pluripotency of stem cells.

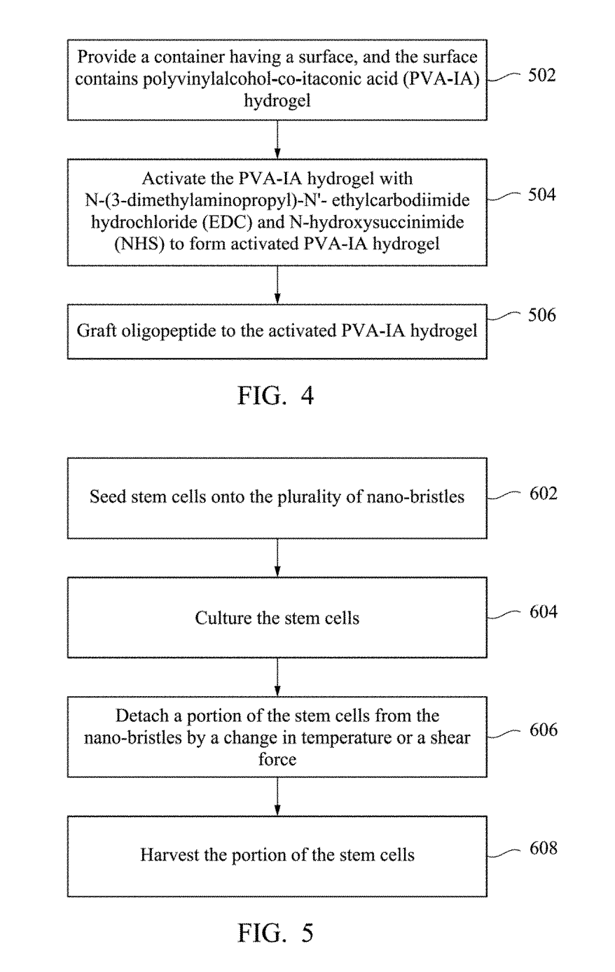

Referring next to FIG. 4, which illustrates a flow chart of forming a cell culture device in accordance with some embodiments of the present disclosure. The method includes: providing a container 100 having a surface 110, and the surface 110 contains the polyvinylalcohol-co-itaconic acid (PVA-IA) hydrogel 112 (step 502), activating the PVA-IA hydrogel 112 with N-(3-dimethylaminopropyl)-N′-ethylcarbodiimide hydrochloride (EDC) and N-hydroxysuccinimide (NHS) to form an activated PVA-IA hydrogel (step 504), and grafting oligopeptide 220 to the activated PVA-IA hydrogel (step 506). The details of each step are elaborated below.

In step 502, a container 100 having a surface 110 that contains PVA-IA hydrogel 112 is provided. PVA-IA (Japan VAM & Poval Co., Ltd., Osaka, Japan) with 0.5-3 mol % itaconic acid showing a degree of hydrolysis of 95-99% and a degree of polymerization of 1500-2000 is dissolved in ultra-pure water at the concentration of 0.02-0.1 wt % for the cell culture or 0.3-1 wt % for the rheometer measurements to form a PVA-IA solution. Then, the PVA-IA solution is agitated for 1-3 days and kept at room temperature for 0.5-2 days to eliminate air bubbles.

Subsequently, 2-5 ml of the PVA-IA solution was added to a container and dried for 5-8 days to produce a PVA-IA film. The PVA-IA film is then Immersed in a crosslinking solution containing 0.5-2 wt % glutaraldehyde, 15-25 wt % sodium sulfate, and 0.5-2 wt % sulfuric acid for 0.2-50 hours to form a crosslinked PVA-IA hydrogel 112. After crosslinking, the PVA-IA hydrogel 112 is washed and kept in ultra-pure water prior to oligopeptide grafting and cell culture, with the ultra-pure water changed twice daily. Sterilization of the PVA-IA hydrogel 112 by immersion in a 75 v/v % ethanol solution overnight and subsequent washing by ultra-pure water are also carried out before the cell culture.

Varying lengths of crosslinking time contributes to varying elasticity (i.e. storage modulus, E′) of the PVA-IA hydrogel. The longer the crosslinking time, the higher the storage modulus, and the stiffer the PVA-IA hydrogels. With the crosslinking time of about 0.2-50 hours, the storage modulus of the PVA-IA hydrogel is about 10-35 kPa.

In step 504, the PVA-IA hydrogel 112 is activated with EDC and NHS to form an activated PVA-IA hydrogel. The PVA-IA hydrogel 112 activation is achieved by immersion in a solution containing 5-15 mg/ml EDC and 5-15 mg/ml NHS for 4-8 hours at 20-30° C. EDC crosslinks the carboxylate group of PVA-IA hydrogel 112 to NHS, forming an amine-reactive NHS ester, which is not hydrolyzed and thus available for subsequent oligopeptide grafting reactions. After the activation, the activated surface containing the NHS ester is then washed with PBS for 2-5 times.

In step 506, grafting the oligopeptide 220 to the activated PVA-IA hydrogel includes adding a fourth solution containing the oligopeptide 220 onto the activated PVA-IA hydrogel, and the oligopeptide 220 has a concentration of 40-1600 μg/ml in the fourth solution. The oligopeptide 220 is dissolved in PBS to from the fourth solution. The activated PVA-IA hydrogel is immersed in the fourth solution and incubated for 20-28 hours at 0-10° C. After grafting the oligopeptide 220, the oligopeptide-grafted PVA-IA hydrogel is washed with ultra-pure water for 10-14 hours to remove residual oligopeptide, and immersed in ultra-pure water till use for cell culture.

Referring next to FIG. 5, which illustrates a flow chart of a method of partially detaching stem cells from a cell culture device in accordance with some embodiments of the present disclosure. The method includes: seeding stem cells onto the plurality of nano-bristles 200 (step 602), culturing the stem cells (step 604), detaching a portion of the stem cells from the nano-bristles 200 by a change in temperature or a shear force (step 606), and harvesting the portion of the stem cells (step 608). The details of each step are elaborated below.

In step 602, stem cells are seeded onto plurality of nano-bristles 200. In some embodiments, the stem cells are seeded at a concentration of 5,000-20,000 cells/cm² onto the nano-bristles 200 of the cell culture device 10, 20, 30, or 40.

In step 604, the stem cells are cultured to expand the size of the cell population. In some embodiments, the stem cells are cultured in a variety of cell culture mediums such as the Dulbecco's Modified Eagle Medium (DMEM), xeno-free mediums, or feeder-free mediums. Under the nourishment of the medium, the stem cells are incubated at 30-40° C. for 0.5-8 days in an incubator with 2-10% CO₂ provided. When the cell population reaches a 70-90% confluence, the stem cells are ready for cell detachment.

In step 606, a portion of the stem cells are detached from the nano-bristles by a change in temperature or a shear force. In some embodiments, the nano-bristles 200 are made of the styrene copolymer 210, and detaching the portion of the stem cells from the nano-bristles 200 by the change in the temperature includes lowering a temperature of the nano-bristles 200 to 0-20° C. In some embodiments, the nano-bristles 200 are maintained at 0-20° C. for 2-8 hours. Due to increased hydrophilicity of the P[St-NIPAAm] 212 at the declined temperature, a portion of the stem cells detach from the nano-bristles 200, while the rest of the stem cells remain attached.

In some embodiments, the nano-bristles 200 are made of the oligopeptide 220, and detaching the portion of the stem cells from the nano-bristles 200 by the shear force includes shaking the nano-bristles. In some embodiments, the nano-bristles 200 are shaken at a rate of 30-100 rpm for 2-10 minutes at the initial culture temperature. Due to elasticity of the PVA-IA hydrogel 112, a portion of the stem cells detach from the nano-bristles 200 while the rest of the stem cells remain attached.

In step 608, the portion of the stem cells are harvested. Whether the portion of the stem cells detaches from the nano-bristles 200 by the change in temperature or the shear force, the detached cells can be harvested for further research of clinical use, while the remaining, attached cells can be once again cultured to expand the cell population. On account of the partial detachment of stem cells, the “culture-detachment” cycle can be repeated in a single cell culture device, resulting in the continuous harvesting of the stem cells.

The stem cells include the human adipose-derived stem cells (hADSCs), the human embryonic stem cells (hESCs), the human induced pluripotent stem cells (hiPSCs), or a combination thereof. The hESC and the hiPSC are also categorized as the human pluripotent stem cells (hPSC).

The following Experimental examples 1-13 and Comparative examples 1-7 are meant to elaborate the specific embodiments of thermoresponsive surfaces of the present disclosure in details, and the following Experimental examples 14-25 and Comparative examples 2, 4, 8-10 are meant to elaborate the specific embodiments of PVA-IA-oligopeptide hydrogels of the present disclosure in details, both to facilitate those skilled in the art to implement the present disclosure. The following Experimental examples and Comparative examples are not meant to limit the present disclosure.

### Experimental Examples 1-3 and Comparative Examples 1-2

Partial Detachment of hADSCs from Thermoresponsive Surfaces

Human adipose-derived stem cells (hADSCs) were initially plated onto tissue culture polystyrene dishes (TCPS) and cultured for 3 passages. After passage 3, the hADSCs were used for culture on different thermoresponsive surface coated with styrene copolymers. The ADSCs after passage 3 were seeded at a concentration of approximately 10,000 cells/cm² onto the thermoresponsive surfaces.

The attachment ratio was calculated using the following equation:

Attachment ratio (%)=(Attached cell number on the surface/Initial seeding cell number)×100%

The detachment ratio was calculated using the following equation:

Detachment ratio (%)=(Detached cell number from the surface/Attached cell number on the surface)×100%

The differentiation ratio was calculated using the following equation:

Differentiation ratio=Percentage of differentiated cells (%)+Percentage of partially differentiated cells×0:5(%)

The hADSCs were seeded onto the thermoresponsive surfaces with different surface density of P[St-AA] and different weight ratios of P[St-NIPAAm] to P[St-PEGMA]. Since the saturated density of coated P[St-AA] was 500 mg/cm², the density was indicated as the 100% P[St-AA] coverage, which was achieved when the concentration of the P[St-AA] is 3 mg/ml in the first solution. Based on this, surface densities of coated P[St-AA] and their respective corresponding percentage of P[St-AA] coverage were 500 mg/cm² for 100%, 375 mg/cm² for 75%, 250 mg/cm² for 50%, and 125 mg/cm² for 25%. Also, since the the oligoVN was herein grafted to the P[St-AA], the percentage of P[St-AA] coverage was also viewed as the percentage of P[St-AA]-oligoVN coverage.

The hADSC were seeded onto the thermoresponsive surface with 25% P[St-AA]-oligoVN coverage ratio and 1:0, 4:1, 7:3, and 0:1 weight ratios of P[St-NIPAAm]:P[St-PEGMA] (N:P), which are respectively Experimental example 1 in (a) and (f), Experimental example 2 in (b) and (g), Experimental example 3 in (c) and (h), and Comparative example 1 in (d) and (i). In addition, some ADSCs were seeded onto the uncoated TCPS, which is Comparative example 2 in (e) and (j). The hADSCs in all groups were cultured for 1 day at 37° C.

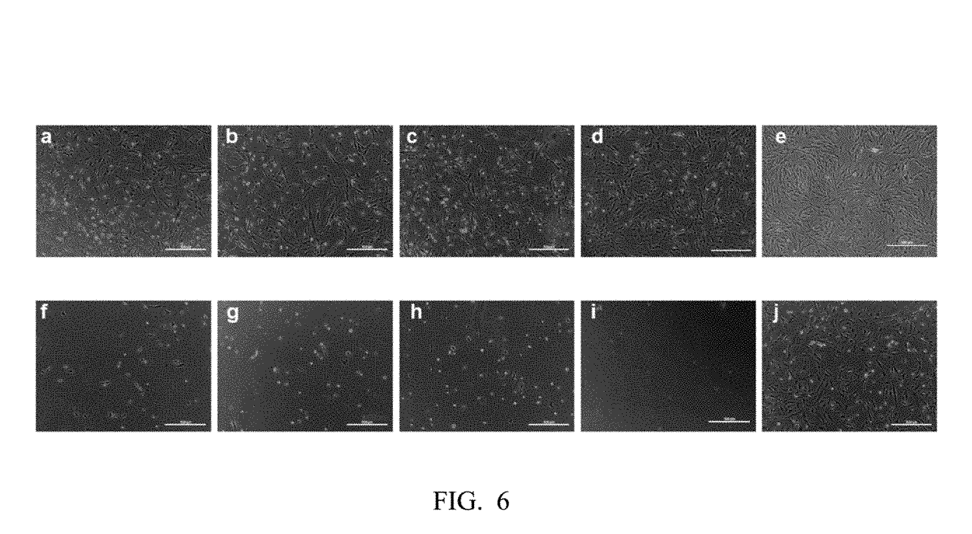

FIG. 6 illustrates hADSC morphologies following culture on thermoresponsive surfaces in accordance with Experimental examples 1-3 and Comparative examples 1-2 of the present disclosure. The bar represents 500 μm. As shown in FIG. 6(a)-(e), regardless of the ratio of P[St-NIPAAm]:P[St-PEGMA] (N:P ratio), hADSCs attached well to Experimental examples 1-3 and showed significant cell expansion, which is similar to ADSCs on Comparative examples 1-2. In (f)-(i) of FIG. 6, after reducing the temperature to 4° C. for 6 hours, the majority of hADSCs partially detached from Experimental examples 1-3 while nearly totally detached from Comparative example 1, indicating partial detachment of ADSCs was achieved in Experimental examples 1-3 with the detachment ratio of 50-90%. However, virtually no hADSCs detached from Comparative example 2.

### Experimental Example 2

Continuous Harvesting of hADSCs from Thermoresponsive Surfaces

TABLE 1
Temperature control timeline for partial detachment of hADSCs

| | Cycle | | | | |
|---|---|---|---|---|---|
| | 1 | 2 | 3 | 4 | 5 |
| 37° C. | 1 day | 3 days | 7 days | 4 days | 3 days |
| 4° C. | 3 hr | 5 hr | 5 hr | 3.5 hr | 5 hr |

The above Table 1 includes 5 cycles of alternating changes in the temperature. As shown in Table 1, each cycle contains the cell expansion period at 37° C. and the cell detachment period at 4° C. The cell expansion period was gradually elongated with the progress of cycle till cycle 3, while the cell detachment period was kept at 3-5 hours.

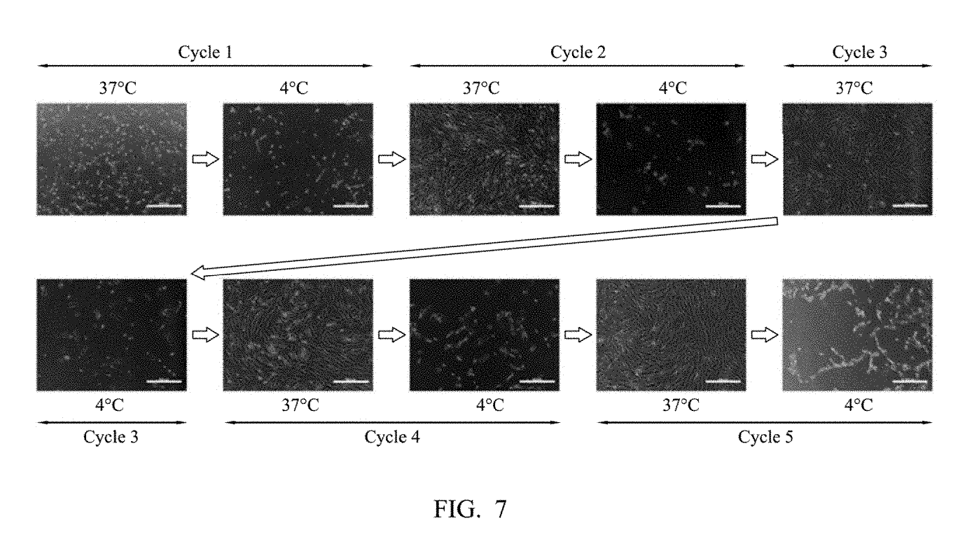

FIG. 7 illustrates morphologies of hADSCs throughout cycles of partial detachment in accordance with Experimental example 2 of the present disclosure. The bar represents 500 μm. As shown in FIG. 7, throughout the 5 cycles, hADSCs could attach well to Experimental example 2 (with 25% P[St-AA]-oligoVN coverage and the N:P ratio of 4:1) at 37° C. and partially detach from Experimental example 2 at 4° C. repeatedly. The remaining, attached hADSCs expanded well on Experimental example 2, which facilitated the continuous harvesting.

### Experimental Examples 1-3

Alterations in Partial Detachment Rates of hADSCs Throughout Continuous Harvesting

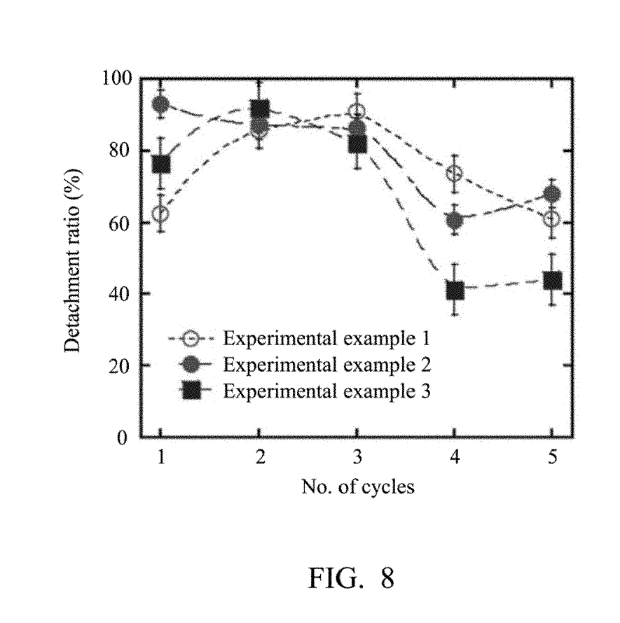

FIG. 8 illustrates detachment ratios of hADSCs throughout cycles of partial detachment in accordance with Experimental examples 1-3 (with the N:P ratio of 1:0, 4:1, and 7:3 respectively) of the present disclosure. The detachment ratios varied throughout the detachment process, exhibiting the trend of an increase in initial cycles and a decrease in latter cycles. In detail, Experimental examples 1 and 2 manifested over 60% detachment rates throughout 5 cycles. In contrast, while coated with the highest amount of P[St-PEGMA], the detachment rate of Experimental example 3 fell to about 40% since cycle 4, indicating that P[St-PEGMA] did not facilitate the repeated detachment of hADSCs for more than four cycles.

### Experimental Example 2

Healthiness of Attached and Detached hADSCs after Continuous Harvesting

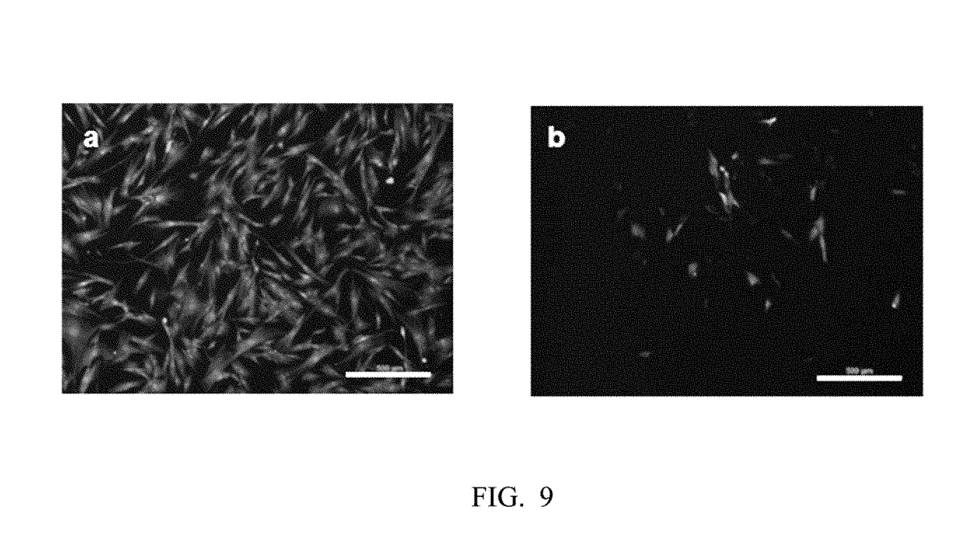

Since hADSCs could be continuously harvested by the change in temperature, verification of the healthiness of the remaining cells so as to undergo repeated temperature-dependent detachment were crucial. To accomplish the assessment, a live/dead staining assay was used to evaluate hADSCs on Experimental example 2 after 5 cycles of partial detachment Living cells were strongly stained. FIG. 9(a) illustrates the hADSCs at 37° C. for 3 days, while FIG. 9(b) illustrates the hADSCs at 4° C. for 5 hours. The bar represents 500 μm. As shown in FIG. 9(a)-(b), hADSCs on Experimental example 2 either before or after partial detachment were all living cells.

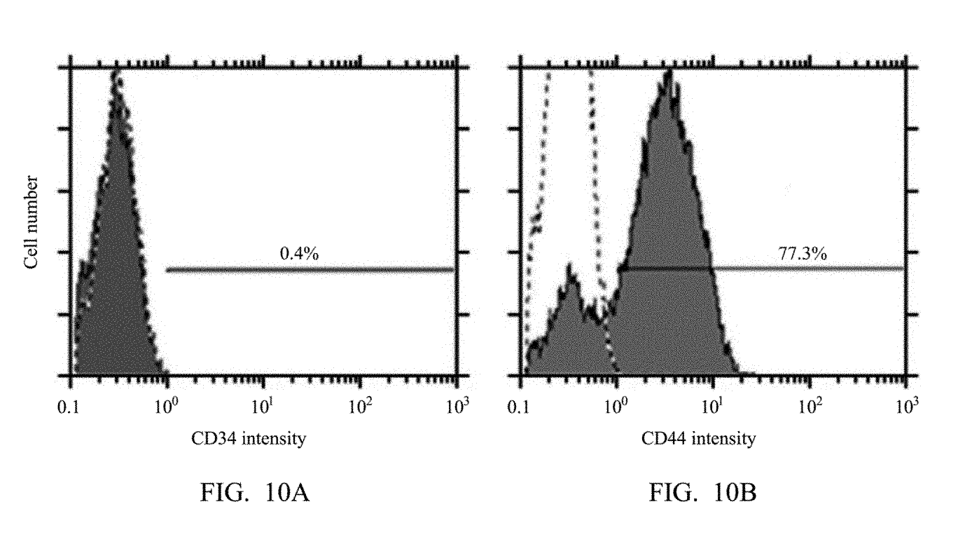

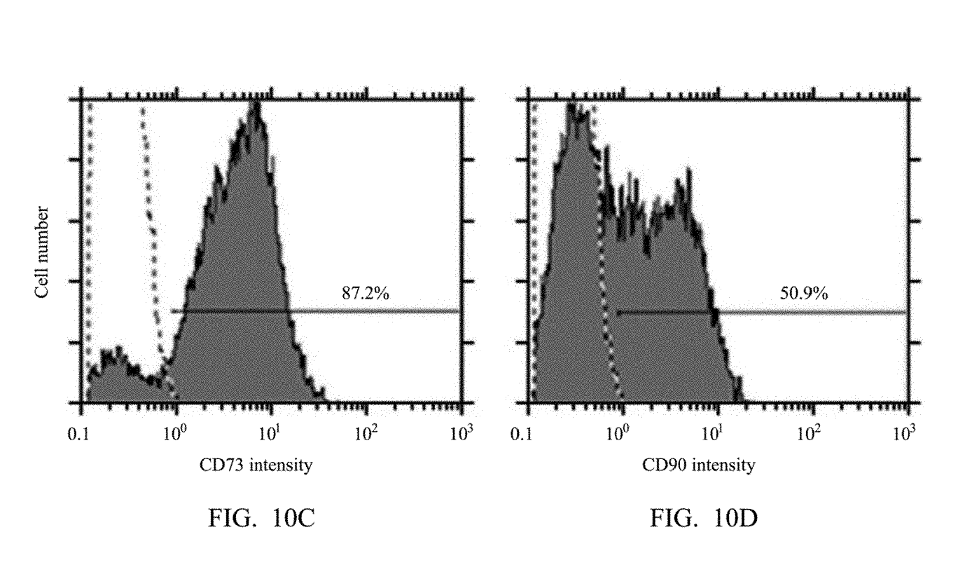

To look into the healthiness of hADSCs detached from Experimental example 2, hADSCs detached after the 5^{th} cycle were stained by antibodies and analyzed by, flow cytometry. FIG. 10A-10D presents the flow cytometry scattergrams for expression analysis of the mesenchymal stem cell (MSC) markers CD44, CD73, and CD90 and the hematopoietic stem cell and endothelial progenitor marker CD34 on hADSCs detached from Experimental example 2. As shown in FIG. 10A-10D, the hADSCs detached from Experimental example 2 strongly expressed MSC surface markers CD44, CD73, and CD90 and did not show CD34 expression. This pattern is similar to the hADSCs cultured on Comparative example 1, which indicate that hADSCs detached from the thermoresponsive surfaces were robust and healthy.

### Experimental Examples 1-2 and Comparative Examples 3.4

Partial Detachment of hESCs from Thermoresponsive Surfaces

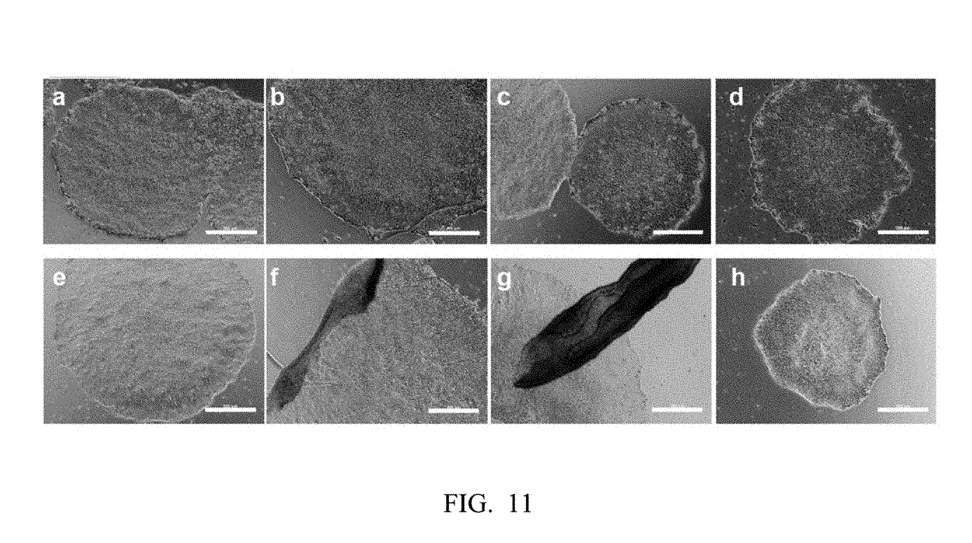

Besides analyzing the hADSCs, the partial detachment of hESCs from the thermoresponsive surfaces were also evaluated. FIG. 11 illustrates morphologies of hESCs cultured on hermoresponsive surfaces in accordance with Experimental examples 1-2 and Comparative examples 3-4 of the present disclosure. The thermoresponsive surfaces for the hESC (the WA09 cell line) culture exhibits 25% P[St-AA]-oligoVN coverage and different N:P ratios. In (a) and (e), the N:P is 0:0 (Comparative example 3). In (b) and (t), the N:P is 1:0 (Experimental example 1). In (c) and (g), the N:P is 4:1 (Experimental example 2). In (d) and (h), hESCs were grown on Matrigel (Comparative example 4). FIG. 11(a)-(d) illustrates hESCs cultured at 37° C. for 5 days, and the FIG. 11(e)-(h) illustrates hESCs cultured at 4° C. for 6 hours.

As shown in FIG. 11(a)-(d), the hESCs cultured at 37° C. were well attached in all groups. In FIG. 11(e)-(h), When the culture temperature was declined to 4° C. for 6 h, hESCs on Experimental examples 1 and 2 showed partial detachment without the need for pipetting. In contrast, hESCs on Comparative examples 3 and 4 didn't show cell detachment following temperature reduction. This indicates that hESCs could be constantly cultured on the thermoresponsive surfaces via partial detachment.

### Experimental Examples 1-2, 4-13 and Comparative Examples 1, 4-7

Attachment, Detachment, and Differentiation of hESCs on Thermoresponsive Surfaces with Different P[St-AA]-oligVN Coverage Ratios and N:P Ratios

To investigate the effect of the P[St-AA]-oligVN coverage ratios and N:P ratios on the attachment, detachment, and differentiation of hESCs, the study included 16 different surfaces defined by the P[St-AA]-oligVN coverage ratios of 25%, 50%, 75%, and 100% coupled to the N:P ratios of 1:0, 9:1, 4:1, and 0:1. Experimental examples 5, 6, 7, and 1 had the N:P ratio of 1:0 and the coverage ratio of 100%, 75%, 50%, and 25% respectively. Experimental examples 8, 9, 10, and 4 had the N:P ratio of 9:1 and the coverage ratio of 100%, 75%, 50%, and 25% respectively. Experimental examples 11, 12, 13, and 2 had the N:P ratio of 4:1 and the coverage ratio of 100%, 75%, 50%, and 25% respectively. Comparative examples 5, 6, 7, and 1 had the N:P ratio of 0:1 and the coverage ratio of 100%, 75%, 50%, and 25% respectively.

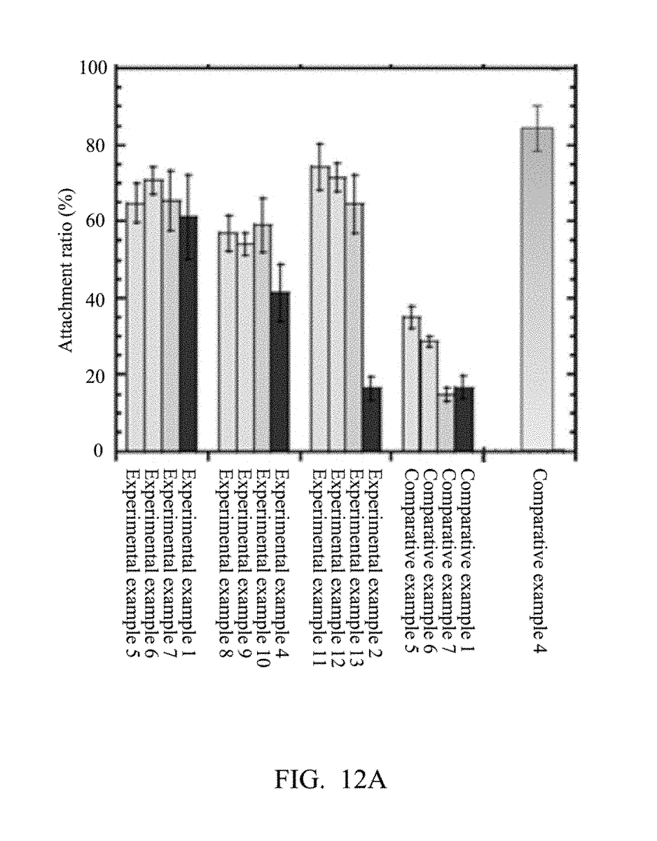

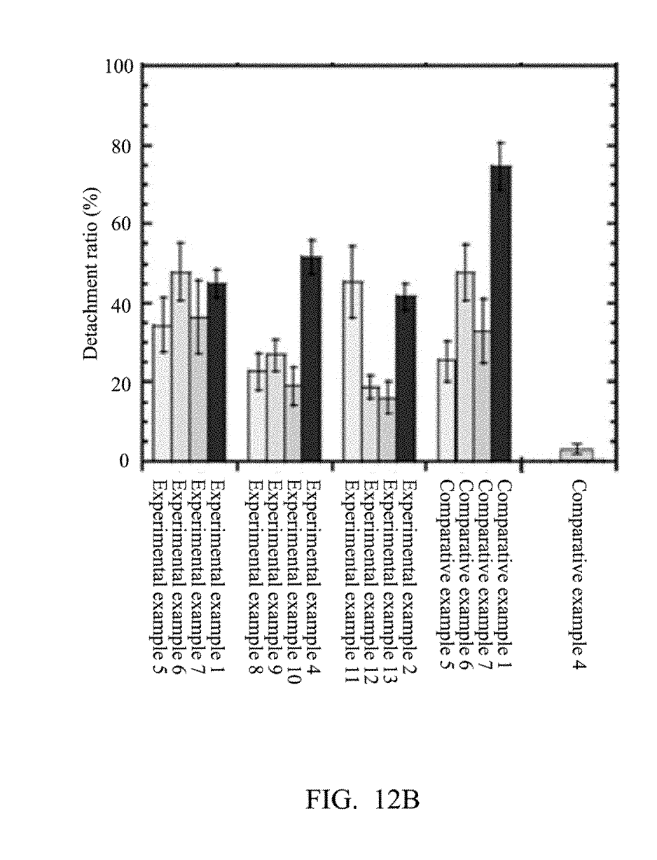

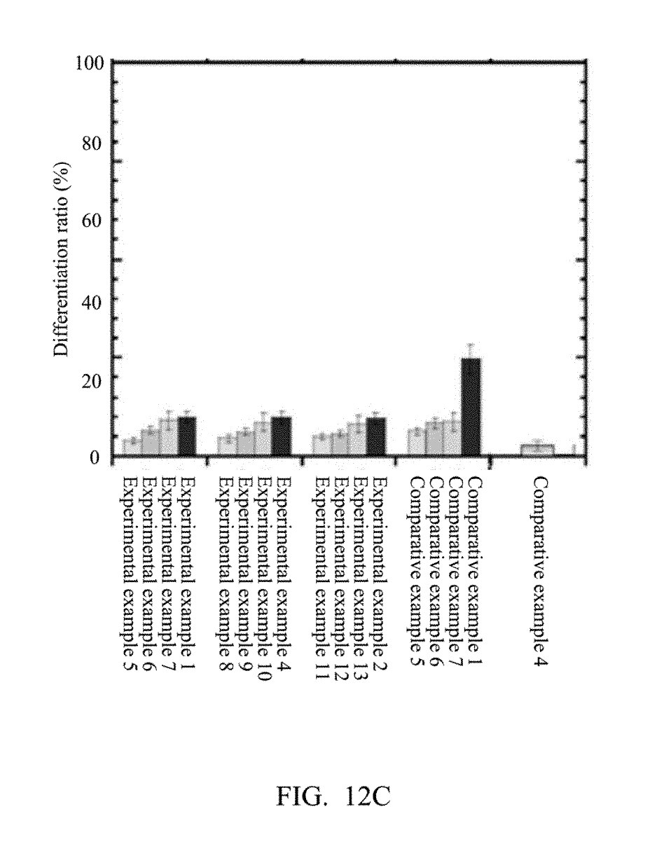

FIG. 12A illustrates attachment ratios of hESCs cultured on thermoresponsive surfaces in accordance with experimental examples 1-2, 4-13 and Comparative examples 1, 4-7 of the present disclosure. As shown in FIG. 12A, experimental examples 5-13 showed high hESC attachment ratios (above 55%). In contrast, experimental example 2, 4 and Comparative example 1 exhibited low hESC attachment ratios (below 40%).

FIG. 12B illustrates detachment ratios of hESCs cultured on thermoresponsive surfaces in accordance with experimental examples 1-2, 4-13 and Comparative examples 1, 4-7 of the present disclosure. As shown in FIG. 12B, detachment ratios of hESCs were relatively low (less than 55%) in all groups except for Comparative example 1. This accounts for that hESCs exhibits a higher affinity for the thermoresponsive surfaces than the hADSCs (with detachment ratios of 50-90%). Moreover, inhomogeneous distribution of the P[St-NIPAAm] across the surfaces might lead to variable hESC detachment. Consequently, despite the increased hydrophilicity of P[St-NIPAAm] at 4° C., it were more difficult for hESCs to detach, achieving both partial detachment and high attachment ratios in Experiment examples 5-13.

FIG. 12C illustrates differentiation ratios of hESCs cultured on thermoresponsive surfaces in accordance with Experimental examples 1-2, 4-13 and Comparative examples 1, 4-7 of the present disclosure. As shown in FIG. 12C, differentiation of hESCs was barely observed in Experimental examples 1-2, 4-13 and Comparative examples 4-7. However, differentiation ratio higher than 3% was observed in Comparative example 1. This indicates that 25% coverage P[St-AA]-oligoVN along with the lack of P[St-NIPAAm]provided insufficient hESC binding sites, which fails to maintain pluripotency.

### Experimental Example 11 and Comparative Example 4

Continuous Harvesting of hESCs from Thermoresponsive Surface

TABLE 2
Temperature control timeline for partial detachment of hESCs

Cycle

| | 1 | 2 | 3 |
|---|---|---|---|
| 37° C. | 5 days | 5 days | 7 days |
| 4° C. | 6 hr | 6 hr | 6 hr |

The above Table 2 includes 3 cycles of alternating changes in the temperature. Each cycle contains the cell expansion period at 37° C. and the cell detachment period at 4° C. The cell expansion period was elongated in cycle 3, while the cell detachment period remain 6 hours throughout the cycles.

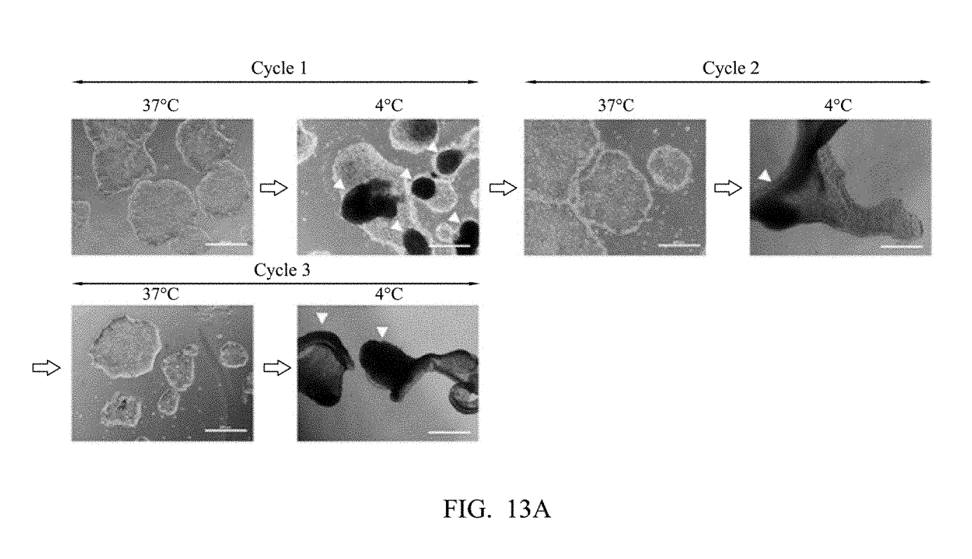

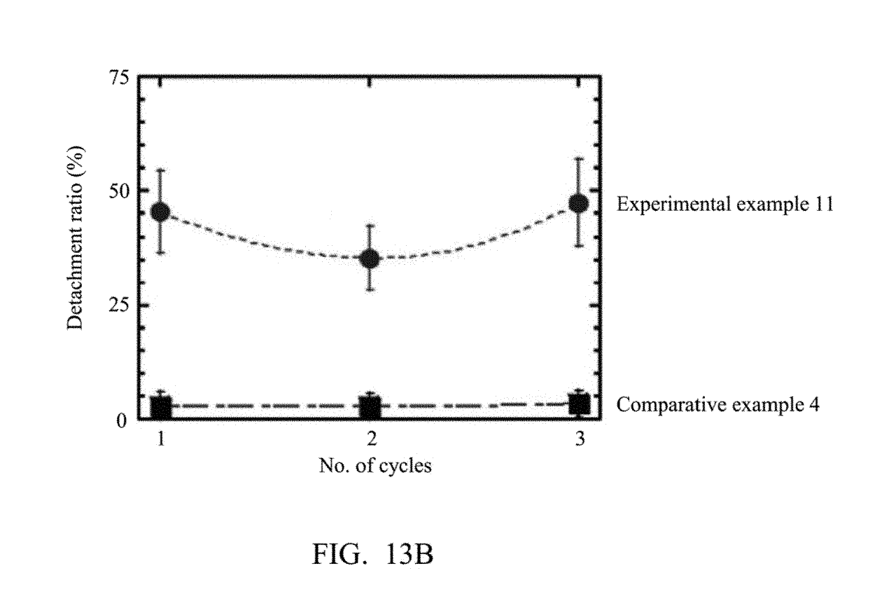

FIG. 13A illustrates morphologies of hESCs throughout cycles of partial detachment in accordance with Experimental example 11 of the present disclosure. The arrows indicate detached hESC colonies. The bar represents 500 μm. As shown in FIG. 13A, throughout the 3 cycles, hESCs of Experimental example 11 (with 100% P[St-AA]-oligoVN coverage and the N:P ratio of 4:1) could attach well to Experimental example 11 at 37° C. and partially detach from Experimental example 11 at 4° C. repeatedly, which facilitates continuous harvest of hESCs.

FIG. 13B illustrates detachment ratios of hESCs throughout cycles of partial detachment in accordance with Experimental example 11 and Comparative example 4 of the present disclosure. The detachment ratios of Experimental example 11 showed a decrease in cycle 2 and a bounce in cycle 3, with all detachment ratios above 25%. Conversely, Comparative example 4 (the Matrigel surface) exhibited nearly no hESC detachment throughout the cycles.

### Experimental Example 11 and Comparative Example 4

Pluripotency and Differentiation Ability of hESCs after Continuous Harvesting

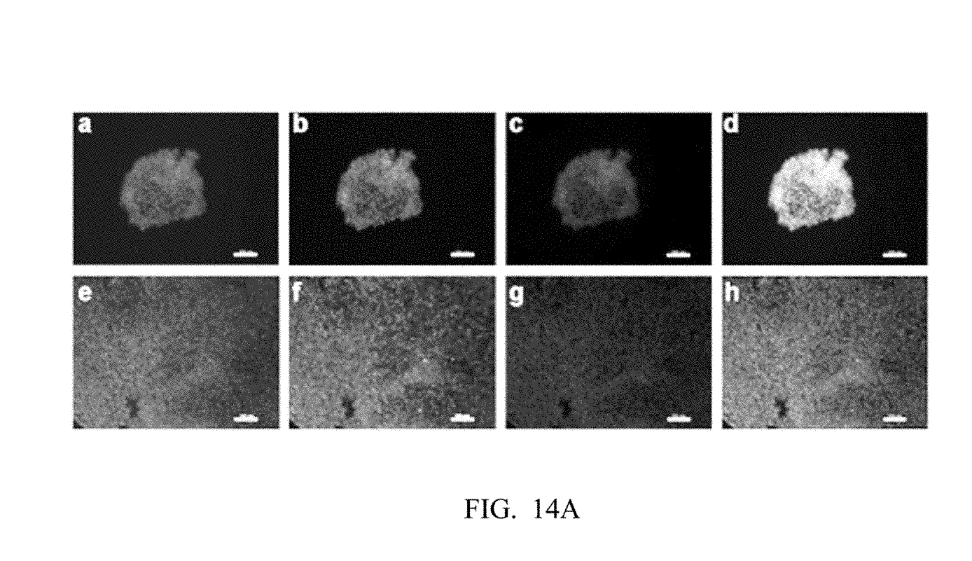

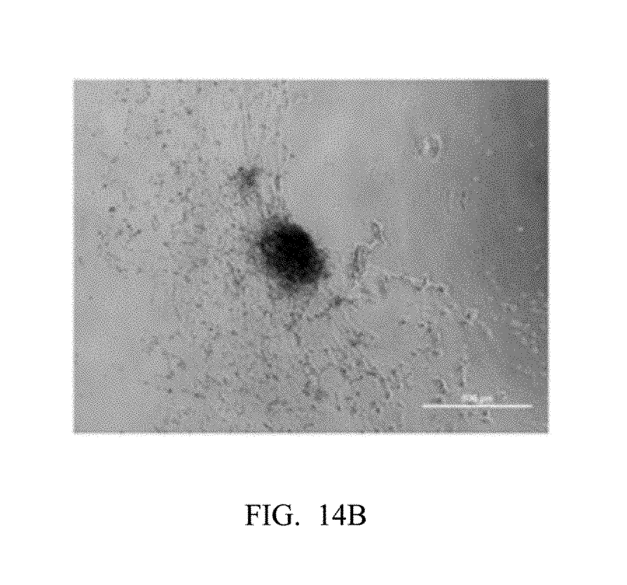

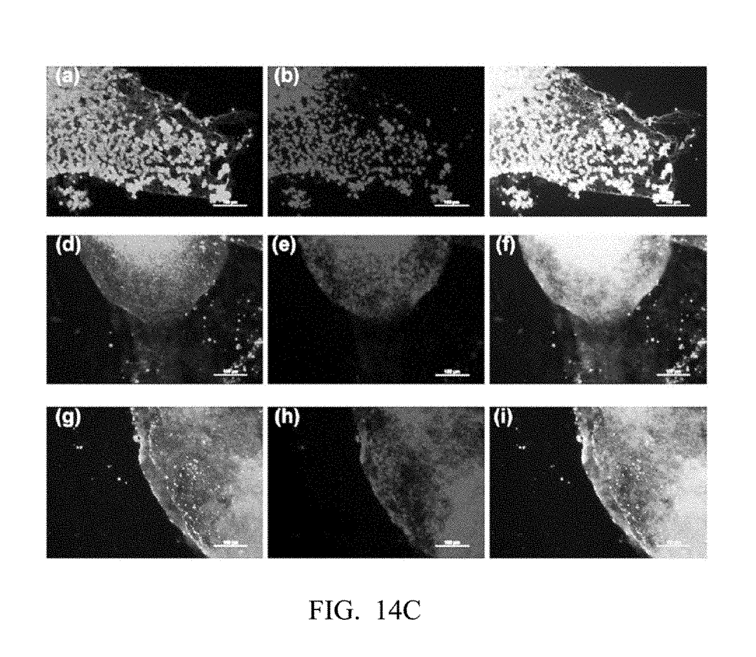

To investigate the pluripotency of hESCs after continuous harvesting, hESCs detached from Experimental example 11 after three partial detachment cycles were immunostained to examine the expression of pluripotency markers octamer-binding transcription factor 4 (Oct4) and sex determining region Y-box 2 (Sox2). hESCs were also stained by Hoechst to specify the cell nucleus. FIG. 14A (a)-(d) Illustrates the Oct4, Sox2, Hoechst, and merged signals of hESCs detached from Experimental example 11 after three partial detachment cycles respectively. FIG. 14A (e)-(h) illustrates the Oct4, Sox2, Hoechst, and merged signals of hESCs cultured on Comparative example 4 respectively. The bar represents 100 μm.

As shown in FIG. 14A (a)-(d), following three cycles of partial detachment, hESCs strongly expressed Oct4 and Sox2 proteins. In FIG. 14A (e)-(h), hESCs grown on Comparative example 4 also expressed these proteins. This indicates that the pluripotency of hESCs after continuous harvesting is maintained.

To investigate whether the maintained pluripotency corresponded to the differentiation ability, the differentiation ability of hESCs detached from Experimental example 11 after three partial detachment cycles was primarily examined by the ability to form embryoid bodies (EBs). FIG. 14B illustrates embryoid bodies formed by hESCs after cycles of partial detachment in accordance with Experimental example 11 of the present disclosure. As shown in FIG. 14B, an embryoid body was formed by hESCs detached from Experimental example 11 after three partial detachment cycles.

To further look into the differentiated cell types, hESCs detached from Experimental example 11 after three partial detachment cycles were immunostained with the endoderm marker alpha-fetoprotein (AFP), the mesoderm marker smooth muscle actin (SMA), and the ectoderm marker glial fibrillary acidic protein (GFAP).

FIG. 14C illustrates immunostaining images of differentiation marker expressions in hESCs after cycles of partial detachment in accordance with Experimental example 11 of the present disclosure. FIG. 14C (a)-(c) illustrates the AFP, Hoechst, and merged signals respectively. FIG. 14C (d)-(f) illustrates the SMA, Hoechst, and merged signals respectively. FIG. 14C (g)-(i) illustrates the GFAP, Hoechst, and merged signals respectively. The bar represents 100 μm.

The hESCs highly expressed AFP, SMA, GFAP as shown in FIG. 14C (a)-(c), (d)-(f), and (g)-(i) respectively. This suggests that hESCs detached from thermoresponsive surface still retain the ability to differentiate into cells derived from all three germ layers (the endoderm, mesoderm, and ectoderm) following continuous harvesting.

### Experimental Example 14 and Comparative Example 8

Bonding Between oligoVN and PVA-IA Hydrogel Gel after oligoVN Grafting

In this study, oligopeptide grafted to PVA-IA hydrogel is oligoVN. PVA-IA hydrogels crosslinked for 24 hours were divided into two groups: one group with grafted oligoVN (Experimental example 14) and the other group without grafting (Comparative example 8). The concentration of oligoVN in the fourth solution during grafting was 1000 μg/ml for Experimental example 14. To evaluate the existence and bonding of oligoVN after grafting, both Experimental example 14 and Comparative example 8 were examined by the X-ray photoelectron spectroscopy (XPS).

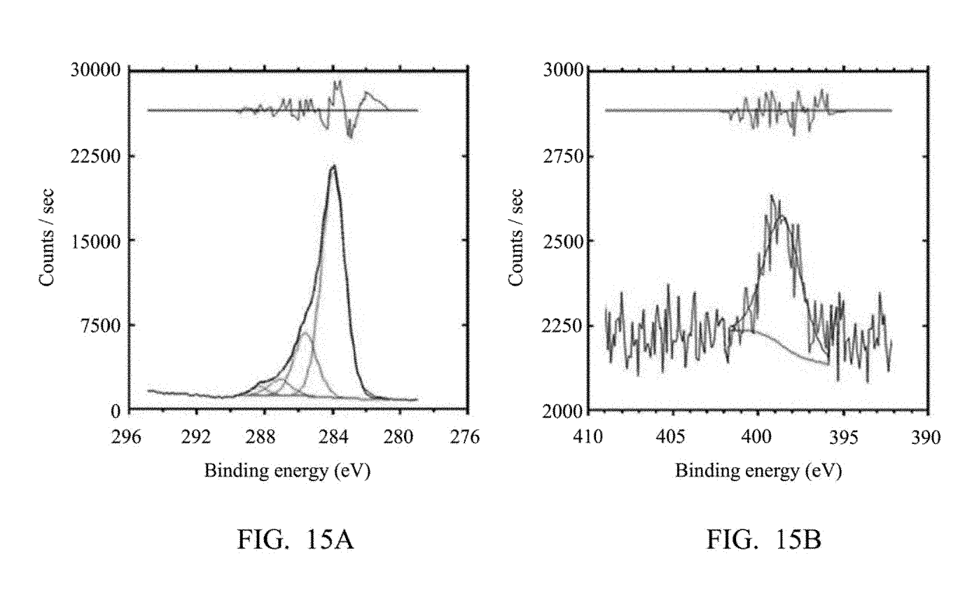

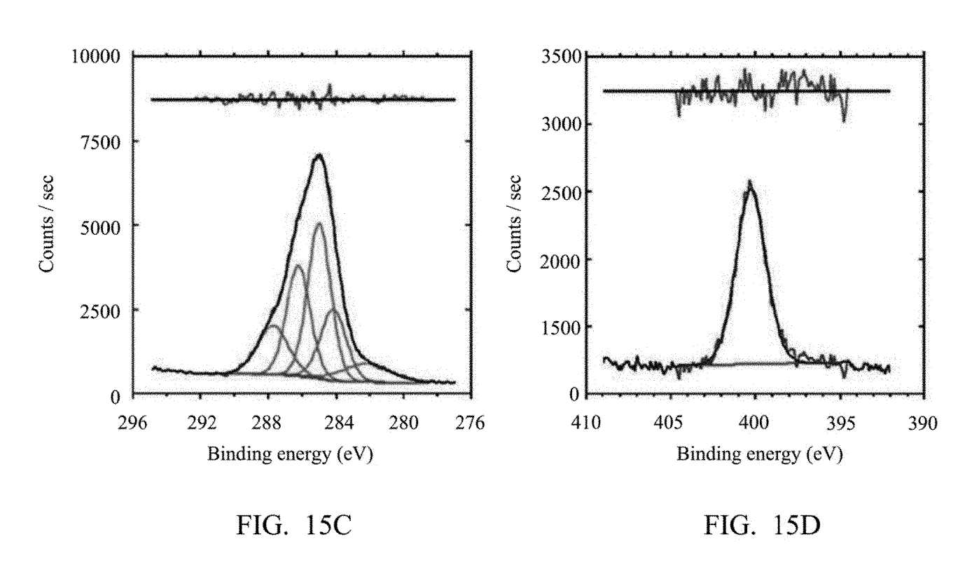

FIG. 15A-15D provides high-resolution XPS spectra of C1s and N1s peaks in accordance with Experimental example 14 and Comparative example 8 of the present disclosure. FIGS. 15A and 15B illustrates the C1s and N1s peaks of Comparative example 8 respectively. FIGS. 15C and 15D illustrates the C1s and N1s peaks of Experimental example 14 respectively. As shown in FIG. 15C, C—N bonding (285.9 eV), O—C═O bonding (289.3 eV), and C—C and C—H bonding (285.0 eV) were clearly observed in the XPS spectra of Experimental example 14. While in FIG. 15A, only the C—C and C—H bonding (285.0 eV) were mainly observed in the XPS spectra of Comparative example 8. As shown in FIG. 15D, a significant N1s peak at 399 eV was observed in the XPS spectra of Experimental example 14. While in FIG. 15B, only a faint N1s peak at 399 eV was found in the XPS spectra of Comparative example 8. Since the PVA-IA hydrogel lacks the nitrogen atom, nitrogen atom is acquired after the oligoVN grafting. The C—N bonding and O—C═O bonding in Experimental example 14 indicates that the oligoVN is covalently conjugated to the PVA-IA hydrogel to form PVA-IA-oligoVN hydrogel.

### Experimental Examples 14-19 and Comparative Examples 2, 9

Effect of oligoVN Concentrations on Surface Densities of Grafted oligoVN

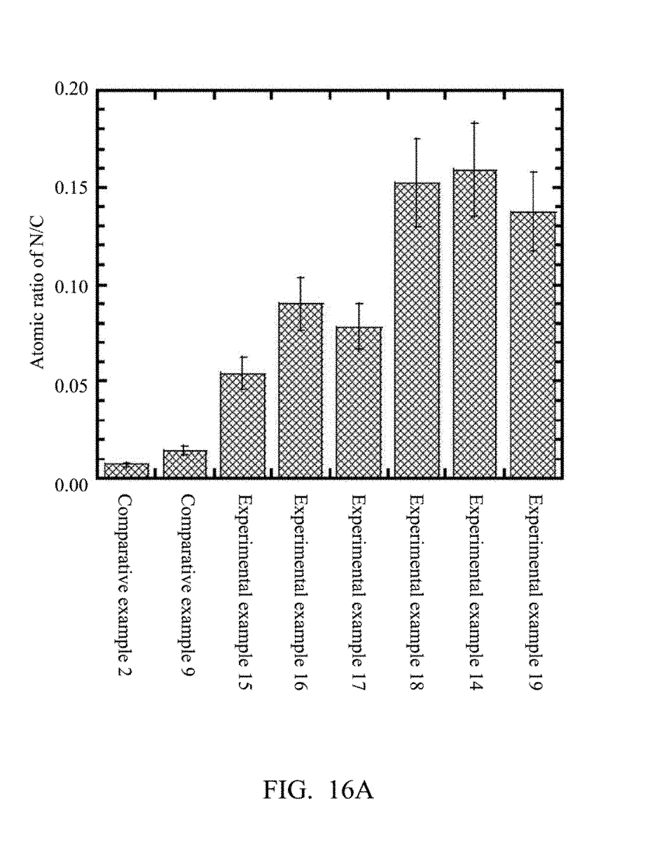

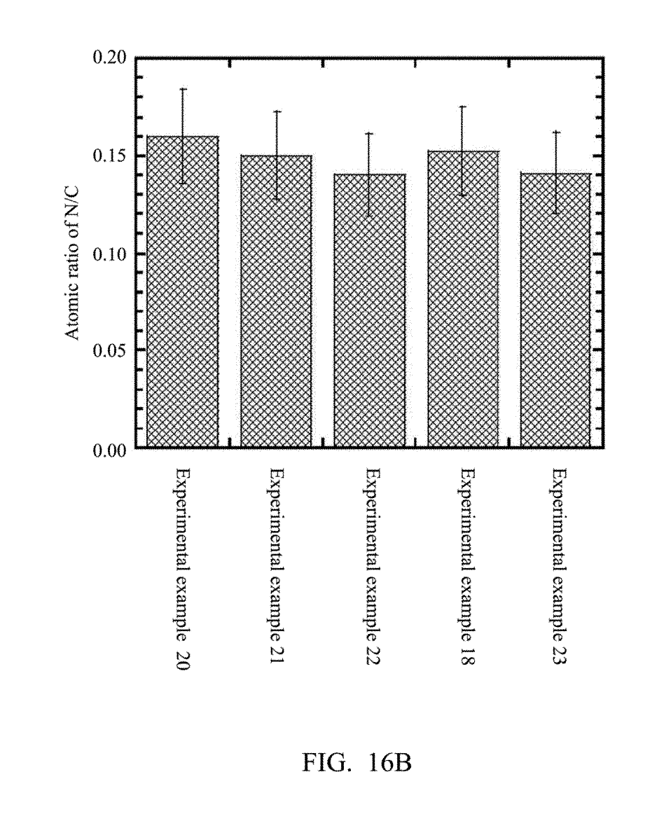

FIG. 16A illustrates atomic ratios of nitrogen to carbon (N/C) of PVA-IA-oligoVN hydrogels under different oligoVN concentrations in accordance with experimental examples 14-19 and Comparative examples 2, 9 of the present disclosure. Experimental examples 15, 16, 17, 18, 14, and 19 represented 24 hr-crosslinked PVA-IA-oligoVN hydrogels with the respective oligoVN concentration of 50, 100, 250, 500, 1000, and 1500 μg/ml during grafting. Comparative example 9 was the 24 hr-crosslinked PVA-IA hydrogel with merely EDC/NHS activation. As shown in FIG. 16A, the N/C ratios of Comparative examples 2 and 9 were minimal. In contrast, N/C ratio increased with the increasing oligoVN concentration up to 500 μg/ml, as Indicated by Experimental examples 15-18. However, when the oligoVN concentration was above 500 μg/ml, the N/C ratios were approximately the same within experimental errors, as indicated by Experimental examples 14 and 19. This suggests that the surface density of oligoVN grafted to the PVA-IA hydrogels becomes saturated when the concentration of oligoVN reaches 500 μg/ml.

### Experimental Examples 18, 20-23

Effect of Different Lengths of Crosslinking Time on Surface Densities of Grafted oligoVN

FIG. 16B illustrates atomic ratios of nitrogen to carbon (N/C) of PVA-IA-oligoVN hydrogels under different lengths of crosslinking time in accordance with experimental examples 18, 20-23 of the present disclosure. Experimental examples 20, 21, 22, 18, and 23 were PVA-IA-oligoVN hydrogels with the oligoVN concentration of 500 μg/ml during grafting and the crosslinking time of 1, 6, 12, 24 and 48 hours respectively. As shown in FIG. 16B, the N/C ratios of Experimental examples 18, 20-23 were approximately the same. Since variation in the crosslinking time gives rise to variations in the elasticity of the PVA-IA-oligoVN hydrogels, this suggests that the surface density of oligoVN grafted to the PVA-IA hydrogels remains approximately the same regardless of the elasticity of the PVA-IA-oligoVN hydrogels.

### Experimental Examples 18, 20-23 and Comparative Examples 4, 10

Attachment and Differentiation of hPSCs on PVA-IA-Oligopepetide Hydrogels with Different Elasticities

TABLE 3
Elasticity of the PVA-IA-oligoVN hydrogels
with varying crosslinking time

| | Crosslinking time | | | | |
|---|---|---|---|---|---|
| | 1 hr | 6 hr | 12 hr | 24 hr | 48 hr |
| Storage | 10.3 kPa | 15.8 kPa | 21.2 kPa | 25.3 kPa | 30.4 kPa |
| modulus | | | | | |

The above Table 3 Illustrates elasticities of PVA-IA-oligoVN hydrogels in association with varying crosslinking time. The longer the crosslinking time, the higher the storage modulus, and the stiffer the hydrogel.

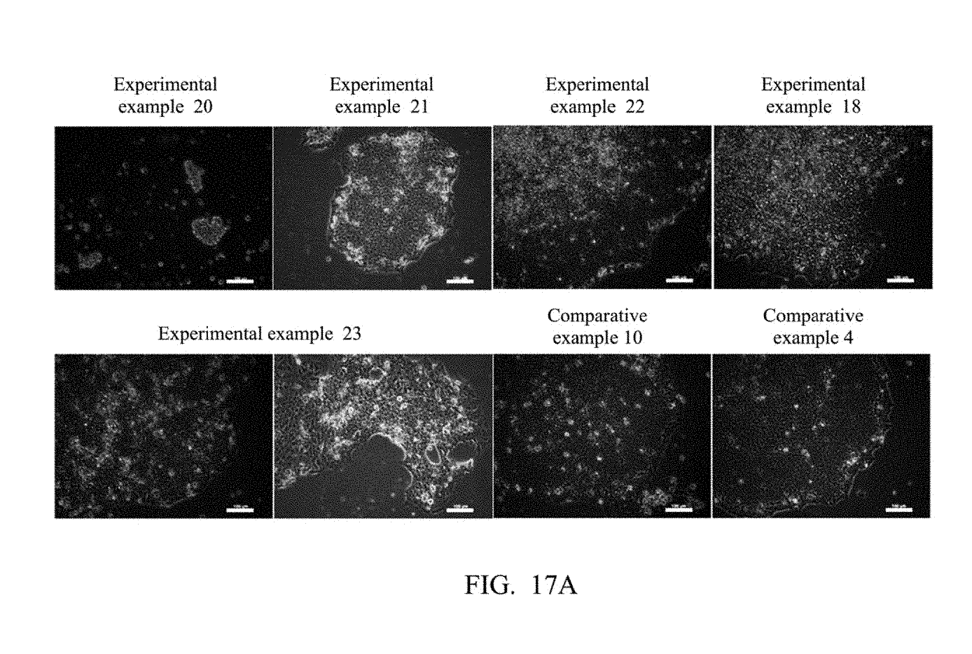

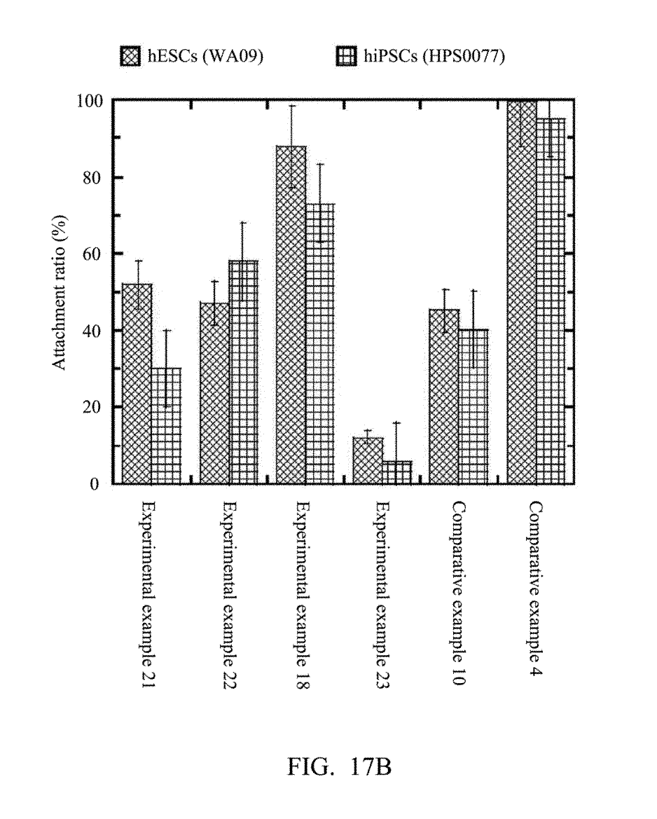

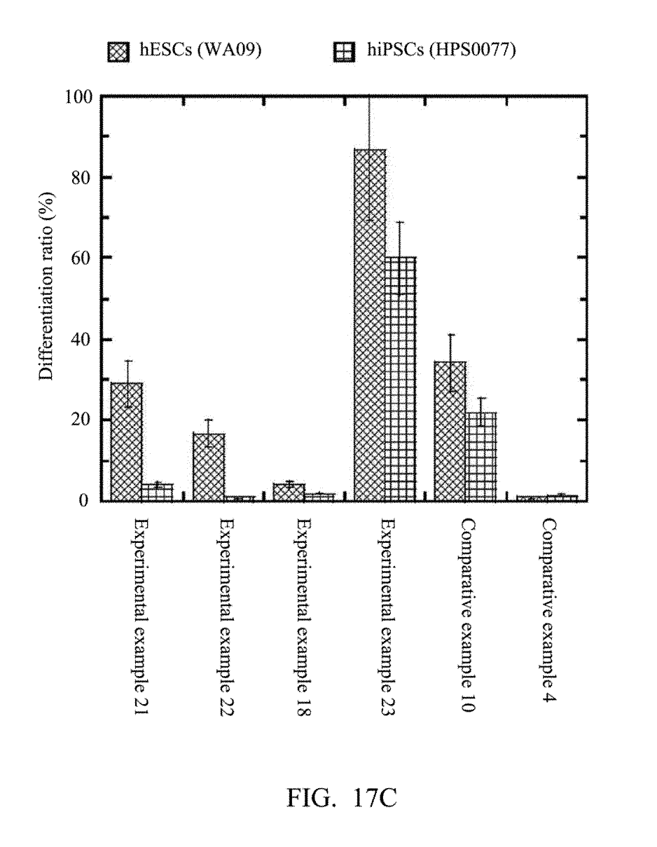

FIG. 17A illustrates morphologies of hESCs cultured on PVA-IA-oligoVN hydrogels with different elasticities in accordance with experimental examples 18, 20-23 and Comparative examples 4, 10 of the present disclosure. Comparative examples 10 represented the Synthemax II surface, while Comparative example 4 represented the Matrigel surface. The bar represents 100 μm. As shown in FIG. 17A, the hESCs (WA09 cell line) at passage 1 did not attach onto Experimental example 20 due to the softness of the hydrogel (with the storage modulus lower than 15 kPa). However, hESCs attached well onto Experimental examples 18, 21-23 due to the storage modulus greater than 15 kPa. This indicates that PVA-IA-oligoVN hydrogels with the storage modulus above 15 kPa is required for hESC attachment.

FIG. 17B illustrates attachment ratios of hESCs and hiPSCs cultured on PVA-IA-oligoVN hydrogels with different elasticities in accordance with Experimental examples 18, 21-23 and Comparative examples 4, 10 of the present disclosure. The hESCs (WA09 cell line) and hiPSCs (HPS0077 cell line) exhibited high attachment ratios on Experimental example 18 and Comparative example 4. Nevertheless, hPSCs showed middle and low attachment ratios on Experimental examples 21-23 and Comparative example 10. This result suggests that PVA-IA-oligoVN hydrogel with optimal elasticity of 25.3 kPa (Experimental example 18) shows the highest attachment ratio among the PVA-IA-oligoVN hydrogels.

FIG. 17C illustrates differentiation ratios of hESCs and hiPSCs cultured on PVA-IA-oligoVN hydrogels with different elasticities in accordance with Experimental examples 18, 21-23 and Comparative examples 4, 10 of the present disclosure. The hESCs (WA09 cell line) and hiPSCs (HPS0077 cell line) exhibited low differentiation ratios on Experimental example 18 and Comparative example 4. Nevertheless, the hESCs and hPSCs showed high differentiation ratios on Experimental example 23 and Comparative example 10. This indicates that the optimal elasticity of 25.3 kPa (Experimental example 18) is necessary to maintain plunpotency.

According to the above attachment and differentiation ratios, the optimal elasticity of PVA-IA-oligoVN hydrogels was defined as 25.3 kPa for both the hESCs and hiPSCs to achieve higher attachment ratios and higher pluripotency (lower differentiation ratios).

### Experimental Examples 14-19 and Comparative Examples 4, 10

Attachment and Differentiation of hESCs on PVA-IA-Oligopepetide Hydrogels with Different Surface Densities of oligoVN

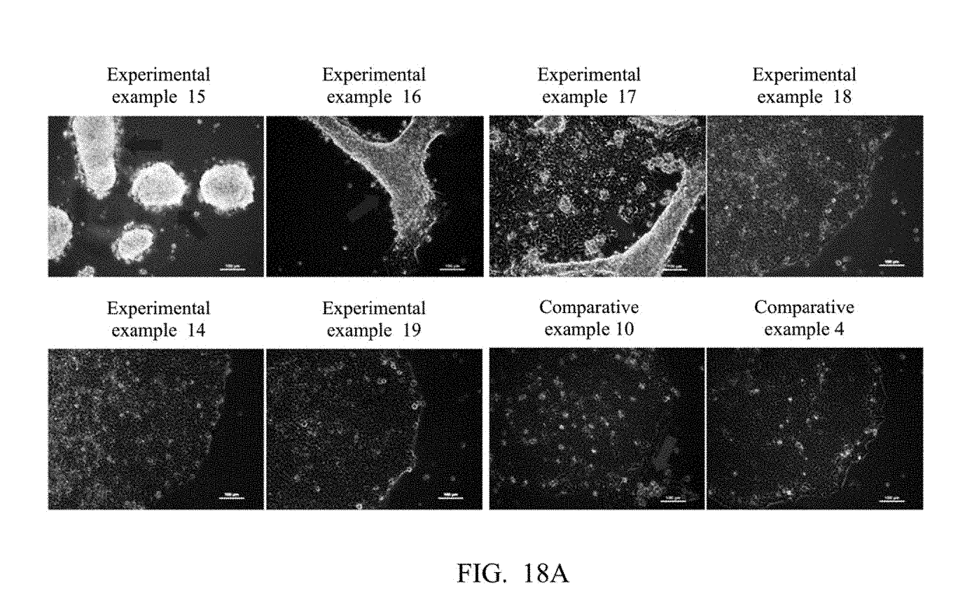

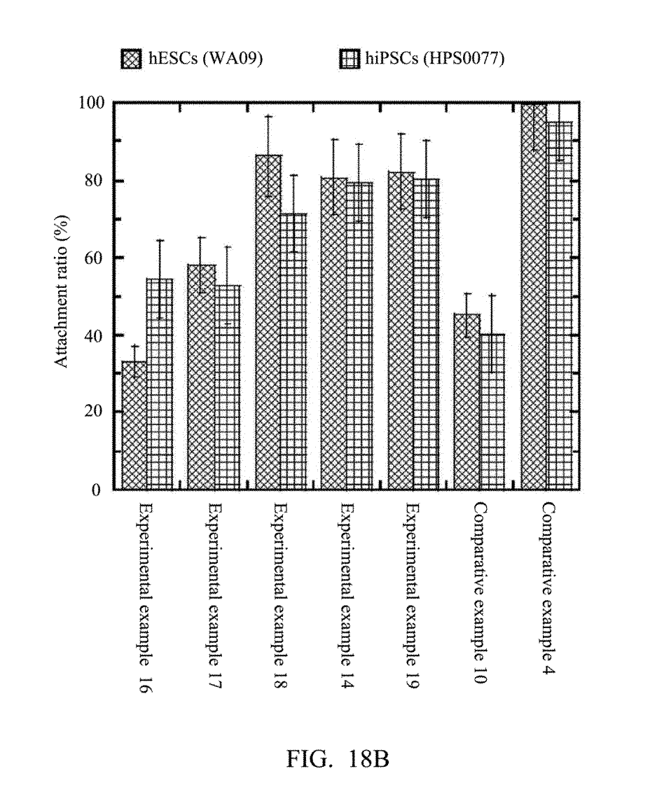

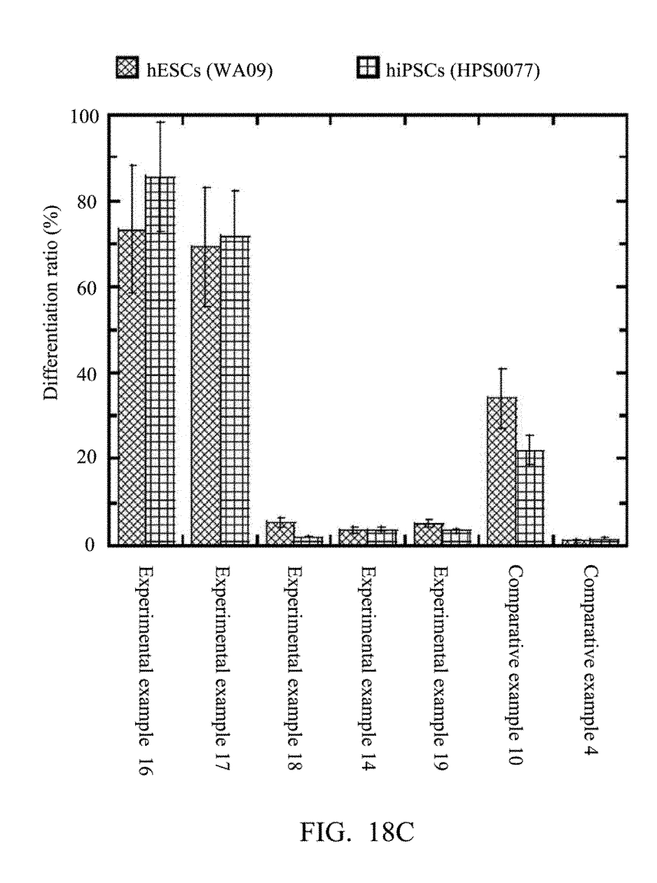

FIG. 18A illustrates morphologies of hESCs cultured on PVA-IA-oligoVN hydrogels with different surface densities of oligoVN In accordance with experimental examples 14-19 and Comparative examples 4, 10 of the present disclosure. Experimental examples 15, 16, 17, 18, 14, and 19 were PVA-IA-oligoVN hydrogels with the crosslinking time of 24 hr and the oligoVN concentration of 50, 100, 250, 500, 1000, 1500 μg/ml respectively during grafting. Comparative examples 10 represented the Synthemax II surface, while Comparative example 4 represented the Matrigel surface. The arrows indicate detached cells. The bar represents 100 μm. As shown in FIG. 18A, the hESCs (WA09 cell line) were found to detach easily from Experimental examples 15-17, in which the oligoVN concentration is less than 500 μg/mi. Moreover, due to poor attachment, the. hESCs could not be cultured on Experimental example 15 for more than 2 passages.

FIG. 18B illustrates attachment ratios of hESCs and hIPSCs cultured on PVA-IA-oligoVN hydrogels with different surface densities of oligoVN in accordance with experimental examples 14, 16-19 and Comparative examples 4, 10 of the present disclosure. The hESCs (WA09 cell line) and hiPSCs (HPS0077 cell line) at passage 3 exhibited increased attachment ratios with increasing concentrations of oligoVN up to 500 μg/ml, as indicated by Experimental examples 16-18. Once the oligoVN concentration reached 500 μg/ml, the attachment ratio of hESCs and hiPSCs remained roughly the same within experimental errors, as indicated by Experimental examples 14 and 19. The attachment ratios of hESCs and hiPSCs on Experimental examples 14 and 19 were significantly higher than those on Comparative example 10, but slightly lower than those on Comparative example 4. This indicates that the hESC and hiPSC attachment is saturated at the oligoVN concentration of 500 μg/ml.

FIG. 18C illustrates differentiation ratios of hESCs and hiPSCs cultured on PVA-IA-oligoVN hydrogels with different surface densities of oligoVN in accordance with experimental examples 14, 16-19 and Comparative examples 4, 10 of the present disclosure. The hESCs (WA09 cell line) and hiPSCs (HPS0077 cell line) at passage 3 exhibited extremely low differentiation ratios on Experimental examples 18, 14, 19 and Comparative example 4.

Nevertheless, hPSCs showed high differentiation ratios on Experimental examples 16-17. This indicates that the threshold oligoVN concentration to maintain the pluripotency of hESCs and hiPSCs is 500 μg/ml.

Based on the hPSC attachment and differentiation ratios, the oligoVN concentration during grafting should be above 500 μg/ml (such as 500-1500 μg/ml) for both the hESCs and hiPSCs to attach well to the PVA-IA-oligoVN hydrogels and retain high pluripotency.

### Experimental Example 14 and Comparative Examples 4, 10

Long-Term Culture of hESCs and hiPSCs on PVA-IA-oligoVN Hydrogels with an Optimal Elasticity Under Xeno-Free Culture Conditions

To evaluate cultures of hESCs and hiPSCs on PVA-IA-oligoVN hydrogels throughout several passages, hESCs (WA09 cell line) and hiPSCs (HPS0077 cell line) were cultured on Experimental example 14 (with 24 hours of crosslinking, the oligoVN concentration of 1000 μg/ml, and the optimal elasticity of 25.3 kPa), Comparative example 4 (the Matrigel surface) and Comparative example 10 (the Synthemax II surface).

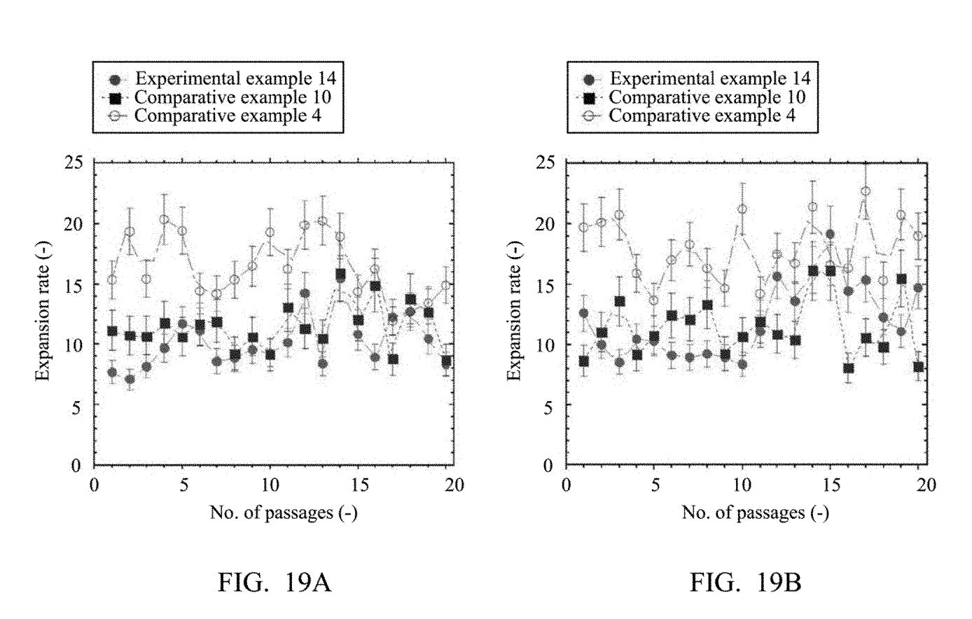

FIG. 19A-19B illustrates expansion rates of hESCs and hiPSCs cultured on PVA-IA-oligoVN hydrogels with an optimal elasticity throughout passages in accordance with Experimental example 14 and Comparative examples 4, 10 of the present disclosure. FIG. 19A presents the expansion rates of hESCs, while and FIG. 19B presents the expansion rates of hiPSCs. As shown in FIG. 19A-19B, the expansion folds of hESCs and hIPSCs cultured on was found to be almost the same as the folds of Comparative example 10, but was slightly lower than the folds of Comparative example 4.

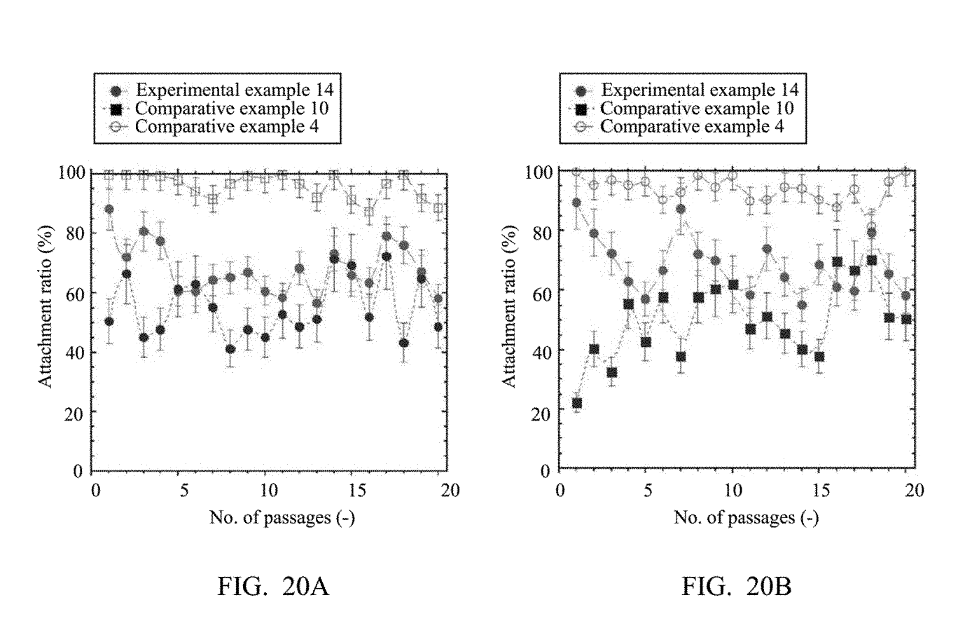

FIG. 20A-20B illustrates attachment ratios of hESCs and hiPSCs cultured on PVA-IA-oligoVN hydrogels with an optimal elasticity throughout passages in accordance with Experimental example 14 and Comparative examples 4, 10 of the present disclosure. FIG. 20A presents the attachment ratios of hESCs, while and FIG. 20B presents the attachment ratios of hiPSCs.

As shown in FIG. 20A-20B, although the expansion folds of hESCs and hiPSCs cultured on Experimental example 14 was not significantly higher than the ratios of Comparative example 10 during 20 passages, there was a significant difference in the early (less than 5) passages. In addition, hESCs and hIPSCs grown on Comparative example 4 showed attachment ratios always higher than 80% throughout 20 passages.

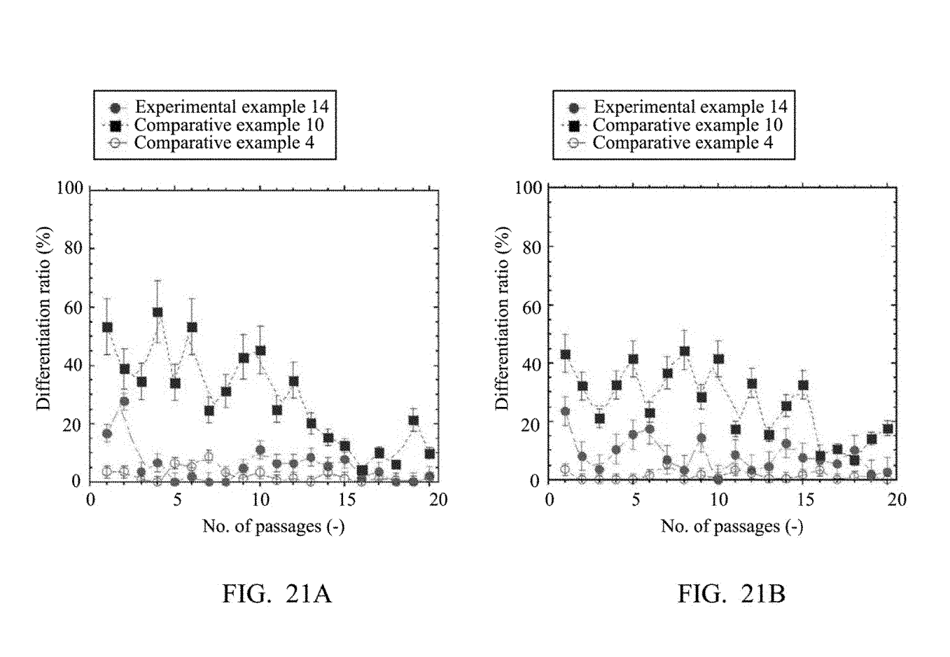

FIG. 21A-21B illustrates differentiation ratios of hESCs and hIPSCs cultured on PVA-IA-oligoVN hydrogels with an optimal elasticity throughout passages in accordance with Experimental example 14 and Comparative examples 4, 10 of the present disclosure. FIG. 21A presents the differentiation ratios of hESCs, while and FIG. 21B presents the differentiation ratios of hiPSCs. As shown in FIG. 21A-21B, the differentiation ratios of hESCs and hiPSCs cultured on Experimental example 14 was as low as ratios of Comparative example 4, which is significant lower than the ratios of Comparative example 10.

According to the above, despite better expansion folds and attachment ratios of the cultures on Comparative example 4, the cultures were under xeno-containing conditions, and thus was preferable for subsequent clinical applications. However, the cultures on Experimental example 14 exhibited higher attachment ratios and lower differentiation ratios over Comparative example 10 under xeno-free conditions. Thus, these results suggest that hESCs and hiPSCs can be cultured on experimental example 14 under xeno-free conditions for several passages.

### Experimental Example 14 and Comparative Example 10

Morphologies of hESCs Cultured on PVA-IA-oligoVN Hydrogels

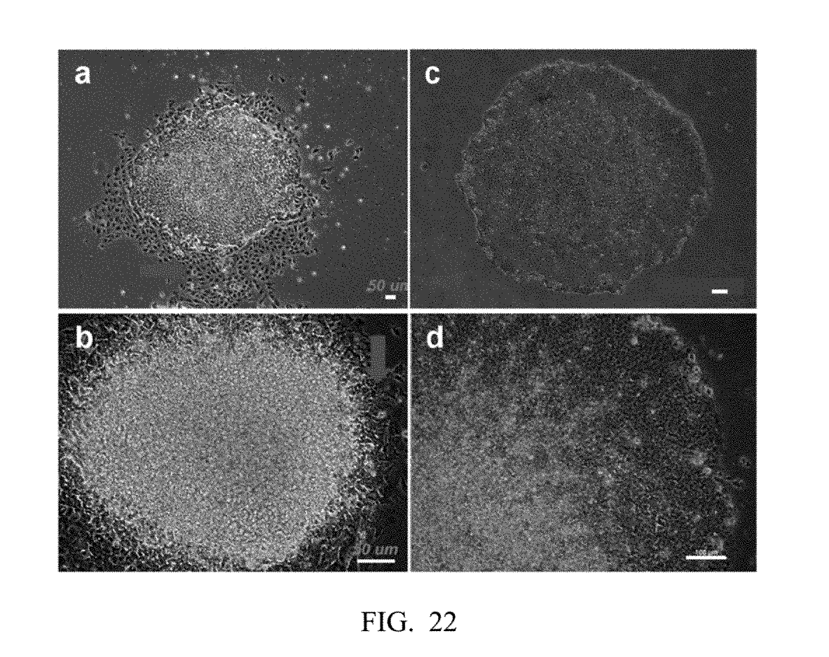

FIG. 22 illustrates colony morphologies of hESCs cultured on PVA-IA-oligoVN hydrogels with an optimal elasticity in accordance with Experimental example 14 and Comparative example 10 of the present disclosure. FIG. 22(a)-(b) presents the hESCs cultured on Comparative example 10, while FIG. 22(c)-(d) presents the hESCs cultured on Experimental example 14. The arrows indicate differentiated hESCs. The bar indicates 50 μm in (a)-(b) and 100 μm in (c)-(d). The hESCs cultured on Comparative example 10 and Experimental example 14 was previously cultured on the mouse embryonic fibroblasts (MEFs) feeder cells in DMEM/Ham's F-12 medium.

As shown in FIG. 22, the hPSCs transferred onto Comparative example 10 differentiated more easily at passage 1, whereas hPSCs transferred onto Experimental example 14 maintained pluripotency. In other words, hESCs on Comparative example 10 manifested more predominant differentiation than those on Experimental example 14.

### Experimental Example 14

Pluripotency of hESCs and hiPSCs Cultured on PVA-IA-oligoVN Hydrogels

The pluripotency of hESCs (WA09 cell line) and hiPSCs (HPS0077 cell line) was evaluated by immunostaining after culturing on Experimental example 14 for 20 passages based on the expression of pluripotent proteins Oct3/4, Sox2, tumor-related antigens-1-81 (Tra-1-81), and stage-specific embryonic antigen-4 (SSEA-4). The hESCs were also stained by Hoechst to the stain the cell nucleus and specify the cell location.

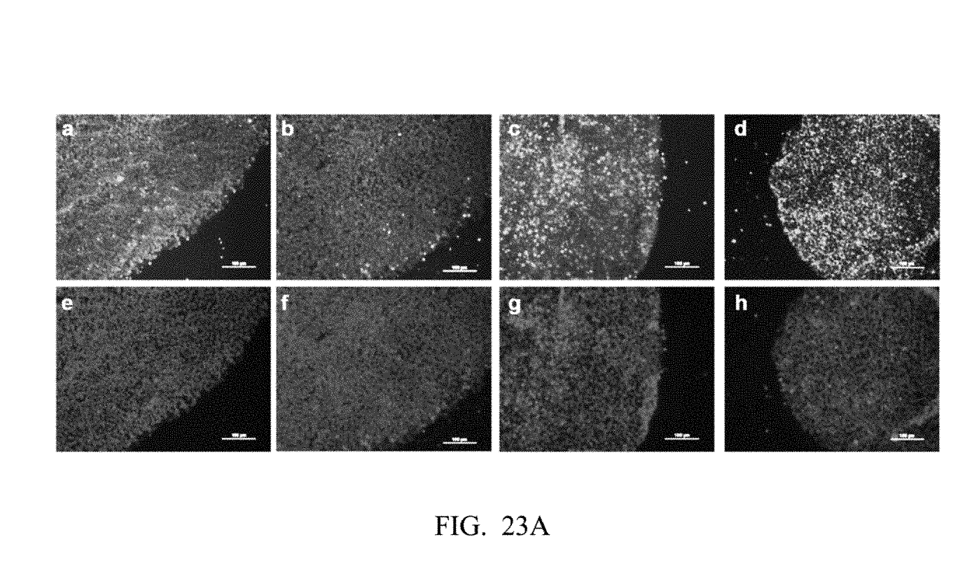

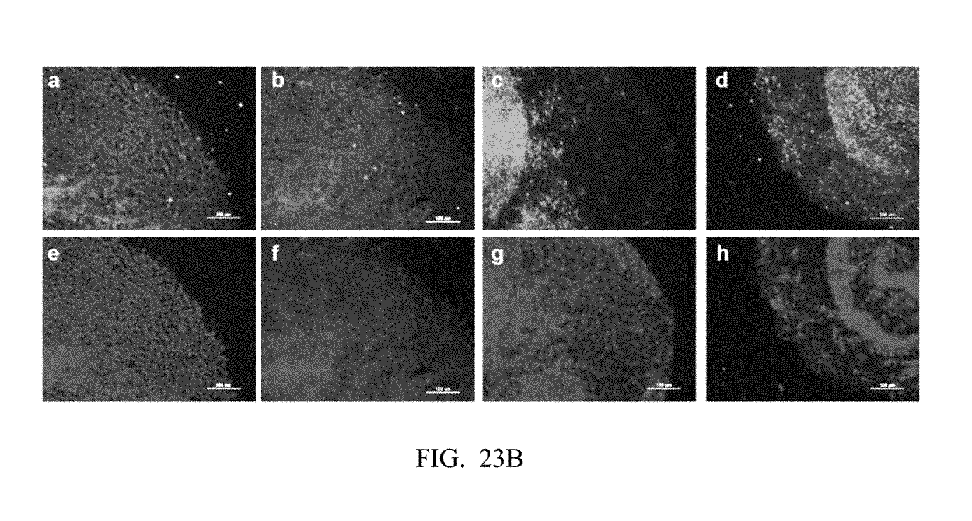

FIG. 23A-23B Illustrates immunostaining images of pluripotency marker expressions of hESCs and hiPSCs cultured on PVA-IA-oligoVN hydrogels in accordance with Experimental example 14 of the present disclosure. FIG. 23A represents the protein expressions of the hESCs, and FIG. 23B represent the protein expressions of the hiPSCs. FIG. 23A (a)-(d) illustrates the Oct3/4, Sox2, Tra-1-81, and SSEA-4 signals of hESCs respectively. FIG. 23A (e)-(h) Illustrates the Hoechst signals of hESCs corresponding to FIG. 23A (a)-(d) respectively. FIG. 23B (a)-(d) illustrates the Oct3/4, Sox2, Tra-1-81, and SSEA-4 signals of hiPSCs respectively. FIG. 23B (e)-(h) Illustrates the Hoechst signals of hiPSCs corresponding to FIG. 23B (a)-(d) respectively. The bar represents 100 μm.

As shown in FIG. 23A-23B, hESCs (WA09 cell line) and hiPSCs (HPS0077 cell line) cultured on Experimental example 14 for 20 passages strongly expressed Oct3/4, Sox2, Tra-1-81, and SSEA-4 under xeno-free conditions, indicating the maintenance of pluripotency.

### Experimental Example 14

In Vitro Differentiation of hESCs and hiPSCs Cultured on PVA-IA-oligoVN Hydrogels

The in vitro differentiation ability of hESCs and hiPSCs was evaluated by embryoid body (EB) formation. After cultured on Experimental example 14 under xeno-free conditions for 20 passages, hESCs (WA09 cell line) and hiPSCs (HPS0077 cell line) were subsequently cultured in suspension using ultra low protein binding dishes to form EBs.

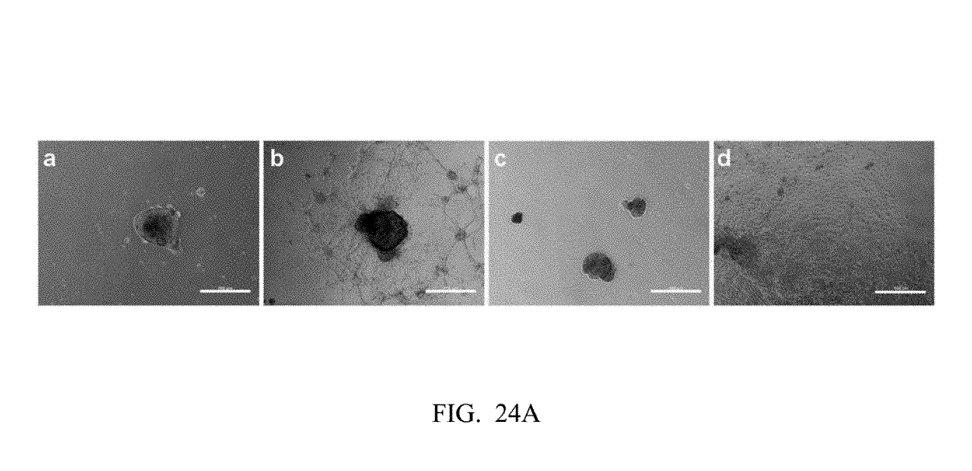

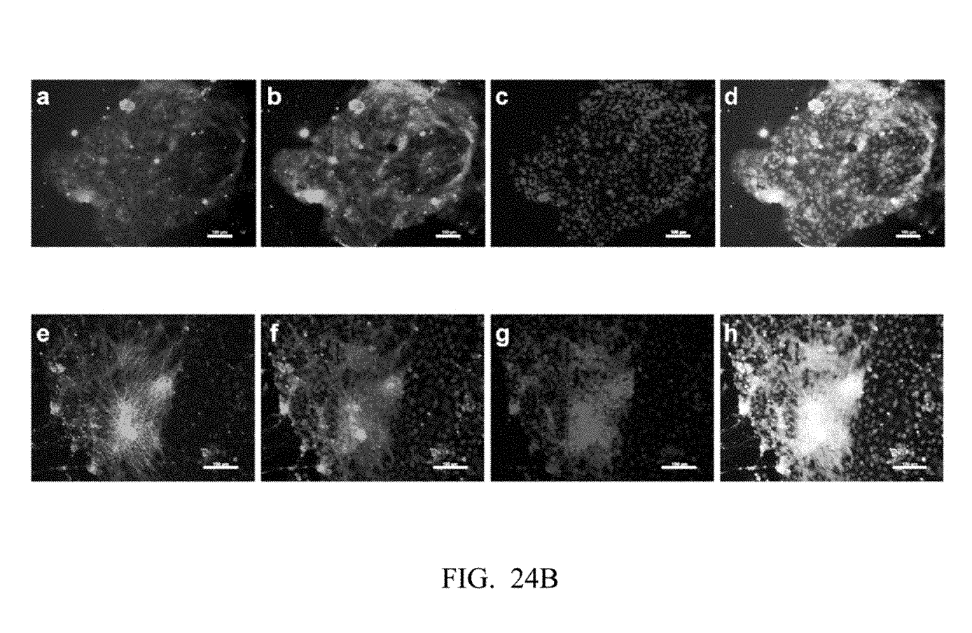

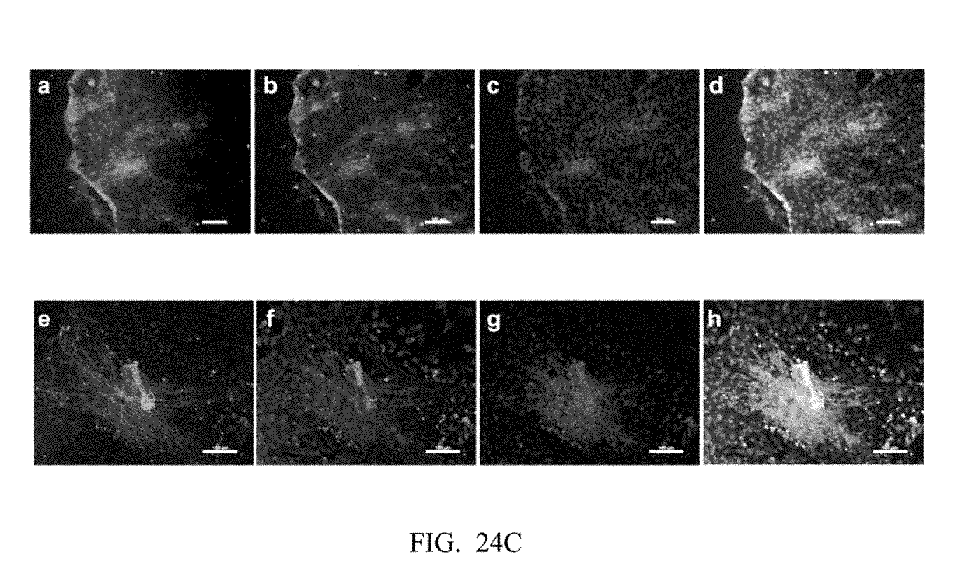

FIG. 24A illustrates embryoid bodies formed by hESCs and hiPSCs cultured on the PVA-IA-oligoVN hydrogel in accordance with Experimental example 14 of the present disclosure. FIG. 24A (a)-(b) represents the embryoid body formed by hESCs (WA09 cell line), and FIG. 24A (c)-(d) represents the embryoid body formed by hiESCs HPS0077 cell line).

After EB formation, the differentiation ability of EBs in vitro was further examined by immunostaining of mesoderm and ectoderm markers. FIG. 24B illustrates mesoderm and ectoderm protein expressions in hESCs cultured on PVA-IA-oligoVN hydrogels in accordance with Experimental example 14 of the present disclosure. FIG. 24B (a)-(d) represents the GFAP (ectoderm marker), SMA (mesoderm marker), Hoechst (nucleus marker), and the merged expression images respectively. FIG. 24B (e)-(h) represents the βIII-tubulin (ectoderm marker), AFP (mesoderm marker), Hoechst (nucleus marker), and the merged expression images respectively. The bar indicates 100 μm. As shown in FIG. 24B (a)-(d), the co-localized expression of GFAP and SMA was observed. In FIG. 24B (e)-(h), the co-localized expression of the βIII-tubulin and AFP was also observed.

FIG. 24C illustrates mesoderm and ectoderm protein expressions by hiPSCs cultured on PVA-IA-oligoVN hydrogels in accordance with Experimental example 14 of the present disclosure. FIG. 24C (a)-(d) represents the GFAP (ectoderm marker), SMA (mesoderm marker), Hoechst (nucleus marker), and the merged expression images respectively. FIG. 24C (e)-(h) represents the βIII-tubulin (ectoderm marker), AFP (mesoderm marker), Hoechst (nucleus marker), and the merged expression images respectively. The bar indicates 100 μm. As shown in FIG. 24C (a)-(d), the co-localized expression of GFAP and SMA was observed. In FIG. 24C (e)-(h), the co-localized expression of the III-tubulin and AFP was also observed.

The results indicates that both hESCs and hiPSCs maintain the pluripotency to differentiate into cells derived from the three germ layers in vitro, including mesodermal cells expressing GFAP and β III-tubulin or ectodermal cells expressing AFP and SMA, after culture on experimental example 14 under xeno-free conditions for 20 passages.

### Experimental Example 14

In Vivo Differentiation of hESCs after Cultured on the PVA-IA-oligoVN Hydrogel

The in vivo differentiation ability of hESCs was evaluated by teratoma formation. The hESCs (WA09 cell line) cultured on Experimental example 14 for 10 passages were subcutaneously xeno-transplanted into non-obese diabetic/severe combined immunodeficiency (SCID) mice to yield teratomas. The teratomas were stained with hematoxylin and eosin (H&E), with the hematoxylin specifying the cell nucleus and the eosin specifying the cytoplasm.

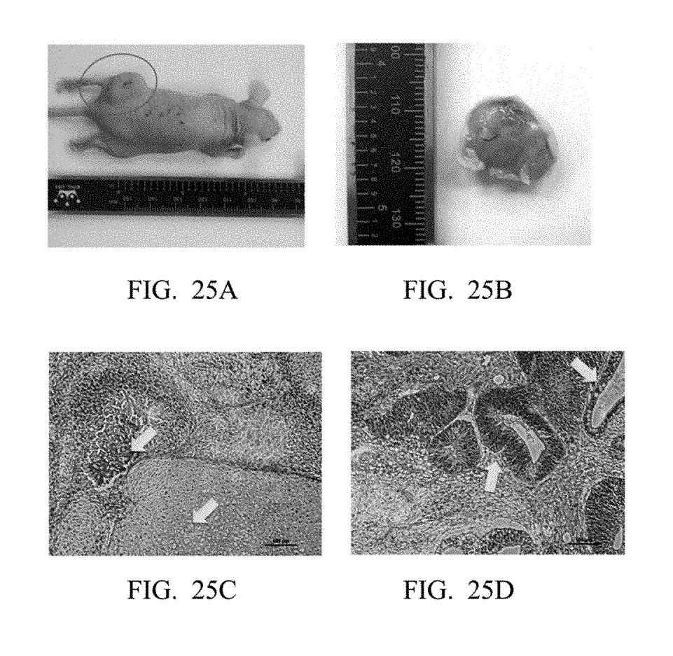

FIG. 25A-25D illustrates the in vivo differentiation of hESCs cultured on the PVA-IA-oligoVN hydrogel in accordance with Experimental example 14 of the present disclosure. The bar indicates 100 μm. The arrows indicate differentiated cells. As shown in FIG. 25A-25B, the hESCs (WA09 cell line) subcutaneously xeno-transplanted into the mouse formed a teratoma (the circled part) after 8 weeks, with a diameter of the teratoma of about 10-15 cm. As shown in FIG. 25C, the H&E staining manifested differentiated cells Including osteoblasts (the upper left arrow) and chondrocytes (the lower right arrow) from the mesoderm. As shown in FIG. 25D the H&E staining manifested differentiated cells including enterons (the upper right arrow) from the endoderm and neurons (the lower left arrow) from the ectoderm.

The above results suggest that long-term culture of hESCs and hiPSCs on Experimental example 14 (with 24 hours of crosslinking, the oligoVN concentration of 1000 μg/ml, and the optimal elasticity of 25.3 kPa) for 10-20 passages can maintain their pluripotency to differentiate into cells derived from the three germ layers in vitro and in vivo.

### Experimental Examples 17, 24, and 25

Detachment Ratios of hESCs Cultured on PVA-IA-Oligopeptide Hydrogels Throughout Partial Detachment Cycles

After realizing the stable growth, maintained pluripotency, and differentiation ability of hESCs on PVA-IA-oligoVN hydrogels for 10-20 passages, it was also of great importance to evaluate the hESCs cultured on the same PVA-IA-oligopeptide hydrogel after several partial detachment cycles.

The hESCs cultured on the PVA-IA-oligopeptide hydrogel could be partially detached by exerting a shear force on the PVA-IA-oligopeptide hydrogel, which was achieved by shaking the cell culture device containing the PVA-IA-oligopeptide hydrogel at 60 rpm for 5 min at the culturing temperature of 37° C. The partial detachment enables the remaining hESCs to expand in population during the subsequent culture on the same PVA-IA-oligopeptide hydrogel, and thus the continuous harvesting of hESCs can be achieved.

TABLE 4
Detachment ratios of hESCs cultured on the PVA-IA-oligopeptide hydrogels

| | Detachment ratio (%) | | | | | | | | | |
|---|---|---|---|---|---|---|---|---|---|---|
| Cycle | 1 | 2 | 3 | 4 | 5 | 6 | 7 | 8 | 9 | 10 |
| Experimental | 85.0 | 80.8 | 87.4 | 85.3 | 78.3 | 83.5 | 88.3 | 91.7 | 89.6 | 80.1 |
| example 17 | | | | | | | | | | |
| Experimental | 87.3 | 85.3 | 91.7 | 81.4 | 89.5 | 88.4 | 80.9 | 83.1 | 86.2 | 83.4 |
| example 24 | | | | | | | | | | |
| Experimental | 89.0 | 86.4 | 91.2 | 83.2 | 86.6 | 90.5 | 86.2 | 87.7 | 82.7 | 85.4 |
| example 25 | | | | | | | | | | |

Table 4 illustrates that hESCs cultured on the four different PVA-IA-oligopeptide hydrogels could be partially detached for 10 cycles. Experimental examples 17, 24, and 25 were dishes containing 24 hr-crosslinked PVA-IA hydrogels with 500 μg/ml of oligoVN, BSP oligopeptide, and VN2C oligopeptide respectively during grafting. Due to the crosslinking time of 24 hr, Experimental examples 17, 24, and 25 all exhibited the elasticity of 25.3 kPa. As shown in the above Table 4, hESCs on Experimental examples 17, 24, and 25 all demonstrated detachment ratios of 78-92% throughout the 10 partial detachment cycles, indicating the detachment ratios of hESCs on Experimental examples 17, 24, 25 were approximately consistent across cycles.

### Experimental Examples 17, 17′, 24, and 26

Expansion Folds of hESCs Cultured on PVA-A-Oligopeptide Hydrogels Throughout Partial Detachment Cycles

Besides the detachment ratios, expansion folds of hESCs cultured on the PVA-IA-oligopeptide hydrogels (including experimental examples 17, 17′, 24, and 25) during each partial detachment cycle were also evaluated.

TABLE 5
Expansion folds of hESCs cultured on the PVA-IA-oligopeptide hydrogels

| Cycle | 1 | 2 | 3 | 4 | 5 | 6 | 7 | 8 | 9 | 10 | sum |
|---|---|---|---|---|---|---|---|---|---|---|---|
| Experimental | 9.8 | 8.5 | 9.4 | 10.2 | 9.5 | 9.3 | 10.3 | 9.5 | 9.7 | 9.2 | 95.4 |
| example 17 | | | | | | | | | | | |
| Experimental | 12.5 | 11.8 | 11.3 | 14.5 | 13.3 | 10.7 | 11.9 | 12.1 | 10.5 | 12.2 | 120.8 |
| example 17′ | | | | | | | | | | | |
| Experimental | 10.0 | 9.3 | 9.5 | 9.7 | 9.7 | 9.2 | 9.4 | 10.2 | 9.3 | 9.6 | 95.9 |
| example 24 | | | | | | | | | | | |
| Experimental | 13.5 | 14.2 | 11.8 | 12.5 | 11.6 | 11.1 | 13.7 | 12.7 | 12.9 | 13.3 | 127.3 |
| example 25 | | | | | | | | | | | |

The difference between Experimental example 17 and 17′ was that Experimental example 17 was the PVA-IA-oligoVN hydrogels on dishes, and Experimental example 17′ was the same PVA-IA-oligoVN hydrogels on microcarriers. As shown in Table 5, hESCs on Experimental examples 17 and 24 showed slightly lower expansion folds, with expansion folds in each cycle lower than 10.5. However, hESCs on Experimental examples 17′ and 24′ showed slightly lower expansion folds, with expansion folds in each cycle no less than 10.5. This indicates that PVA-IA-oligoVN hydrogels on microcarriers and PVA-IA hydrogels grafted with the VN2C oligopeptide are optimal for the hESC culture over cycles of partial detachment and continuous harvesting.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that some modifications and variations can be made to the structure of the present disclosure without departing from the scope or spirit of the disclosure. In view of the foregoing, it is intended that the present disclosure cover modifications and variations of this disclosure provided they fall within the scope of the following claims.

1-16. (canceled)

17. A method of partially detaching stem cells, the method comprising:
receiving a cell culture device, wherein the cell culture device comprising: a container having a surface; and a plurality of nano-bristles immobilized to the surface, wherein the nano-bristles comprises ECM segment-grafted P[St-AA], and P[St-NIPAAm], and the ECM segment is configured to provide binding sites of stem cells:
seeding the stem cells onto the plurality of nano-bristles;
culturing the stem cells;
detaching a portion of the stem cells from the nano-bristles by a change in temperature; and
harvesting the portion of the stem cells.

18. The method of claim 17, wherein:
detaching the portion of the stem cells from the nano-bristles by the change in the temperature comprises lowering a temperature of the nano-bristles to 0-20° C.

19. (canceled)

20. The method of claim 17, wherein the stem cells comprise human adipose-derived stem cells (hADSCs), human embryonic stem cells (hESCs), human induced pluripotent stem cells (hiPSCs), or a combination thereof.

21. The method of claim 17, wherein the ECM segment is vitronectin (VN) oligopeptide.

22. The method of claim 17, wherein the nano-bristles further comprises poly[styrene-co-polyethylene glycol methacrylate] (P[St-PEGMA]).

23. The method of claim 22, wherein a weight ratio of the P[St-PEGMA] to the P[St-NIPAAm] is 0:1 to 3:7.

24. The method of claim 17, wherein the surface comprises polystyrene.

25. The method of claim 17, wherein the container comprises a dish, a flask, or a microcarrier.
